(19) United States
(12) Patent Application Publication
(10) Pub. No.: US 2018/0185347 A1
(43) Pub. Date: Jul. 5, 2018

(54) TETRAHYDROCYCLOPENTA[B]INDOLE COMPOUNDS AND PHOSPHODIESTERASE INHIBITORS FOR THE TREATMENT OF THE SIGNS AND SYMPTOMS OF BHP

(71) Applicant: Transition Therapeutics (Ireland 2) Limited, Dublin 2 (IE)

(72) Inventors: Antonio F. CRUZ, Toronto (CA); Phillip FROST, Miami Beach, FL (US)

(73) Assignee: Transition Therapeutics (Ireland 2) Limited, Dublin 2 (IE)

(21) Appl. No.: 15/814,010

(22) Filed: Nov. 15, 2017

Related U.S. Application Data
(60) Provisional application No. 62/423,159, filed on Nov. 16, 2016.

(51) Int. Cl. A61K 31/4439; A61K 31/519; A61K 31/53; A61K 31/4985; A61K 45/06; A61K 9/48; A61P 13/08

(52) U.S. Cl. CPC A61K 31/4439; A61K 31/519; A61K 31/53; A61K 2300/00; A61K 45/06; A61K 9/48; A61P 13/08; A61K 31/4985

(57) ABSTRACT

Methods of treating the signs and symptoms of Benign Prostatic Hyperplasia (BPH) in a subject by administering at least one tetrahydrocyclopenta[b]indole compound are disclosed. Also disclosed are methods of treating the signs and symptoms of BPH in a subject by administering at least one tetrahydrocyclopenta[b]indole compound in combination with a phosphodiesterase type-5 inhibitor.

### FIELD OF THE INVENTION

Methods of treating the signs and symptoms of Benign Prostatic Hyperplasia (BPH) in a subject by administering at least one tetrahydrocyclopenta[b]indole compound are disclosed. Also disclosed are methods of treating the signs and symptoms of BPH in a subject by administering at least one tetrahydrocyclopenta[b]indole compound in combination with a phosphodiesterase type-5 inhibitor.

### BACKGROUND OF THE INVENTION

Benign Prostatic Hyperplasia (BPH) is a complex pathologic process and progressive disease in aging men that results in the abnormal growth of the prostate. The increase in size and volume of the prostate can result in increased urinary tract symptoms, acute urinary retention and potentially surgery. Drugs reducing the volume of the prostate have been shown to provide favorable improvement in bladder outlet obstruction, peak flow rate and symptom scores. Although the reason for the abnormal growth of the prostate is not well understood, it is thought to be dependent on hormones and growth factors, most notably on testosterone and its more active metabolites. By virtue of the testosterone mechanism of action on prostate growth, lowering the circulating testosterone levels or/and antagonizing the direct effects of testosterone specifically on the prostate should reduce prostate volume and improve the associated distressing urinary tract symptoms. Further, pelvic floor muscles play an important role in incontinence and other urinary functions. It is thought that these muscles begin to atrophy with aging or local trauma contributing to decreased urine flow. Thus, drugs which can increase pelvic flow muscles may also provide benefit in BPH related urinary symptoms. There is a significant need for new medications to treat BPH and a significant need to treat those patients that have not yet been diagnosed with BPH but have some degree of prostate hypertrophy and are on their way to developing BPH.

### SUMMARY OF THE DISCLOSURE

In one aspect, disclosed are methods of treating the signs and symptoms of benign prostatic hyperplasia (BHP) by administering at least one tetrahydrocyclopenta[b]indole compound and/or a combination of the compound with at least one additional active ingredient, wherein the tetrahydrocyclopenta[b]indole compounds have Formula I:

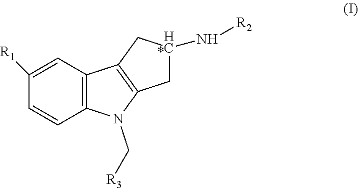

wherein the C* atom may be R, S or R/S configuration (a racemic or diastereomeric mixture);

R₁ represents cyano, —CH═NOCH₃, —OCHF₂, or —OCF₃;

R₂ represents —COR₂ₐ or —SO2R_{2b};

R₂ₐ represents (C₁-C₄)alkyl, (C₁-C₄)alkoxy, cyclopropyl, or —NRaRb;

R_{2b} represents (C₁-C₄)alkyl, cyclopropyl, or —NRaRb;

Ra and Rb each independently is H or (C₁-C₄)alkyl; and

R₃ represents a heteroaryl group selected from the group consisting of pyridinyl, pyrimidinyl, pyrazinyl, pyridazinyl, thiazoloy isothiazolyl, and thiadiazolyl, each optionally substituted with 1 or 2 substituents independently selected from the group consisting of methyl, ethyl, bromo, chloro, fluoro, —CHF₂, —CF₃, hydroxyl, amino and —NHCH₂CO₂H, or a pharmaceutically acceptable salt thereof. U.S. Pat. No. 7,968,587 discloses the compounds of formula I and is incorporated herein by reference.

In another aspect, disclosed methods of treating the signs and symptoms of benign prostatic hyperplasia (BHP) by administering at least one one tetrahydrocyclopenta[b]indole compound having the formula of Formula I in combination a phosphodiesterase type-5 inhibitor, such as tadalafil.

In another aspect, disclosed are methods of treating the signs and symptoms of benign prostatic hyperplasia (BPH) in a subject comprising administering a therapeutically effect amount of the compound of Formula II or a pharmaceutically acceptable salt thereof to a subject in need thereof, wherein the compound has Formula II:

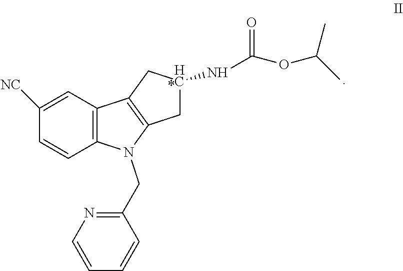

In another aspect, disclosed methods of treating the signs and symptoms of benign prostatic hyperplasia (BHP) by administering Formula II in combination a phosphodiesterase type-5 inhibitor, such as tadalafil.

The compounds disclosed in U.S. Pat. No. 7,968,587 are useful in the treatment of disorders typically treated with androgen therapy. These disorders include hypogonadism, reduced bond mass or density, osteoporosis, osteopenia, reduced muscle mass or strength, sarcopenia, age related functional decline, delayed puberty in boys, anemia, male or female sexual dysfunction, erectile dysfunction, reduced libido, depression, and lethargy. The compounds are described as potent androgen receptor (AR) ligands that agonize the androgen receptor and selectively bind thereto (SARMs). However, the patent does not disclose the use of these compounds to treat BPH. In addition, there is no disclosure therein that such compounds are potent antagonists on the prostate at low doses and lack agonist activity on the prostate even at very high doses. WO 2016040234 discloses the use of (S)-(7-cyano-4-pyridin-2-ylmethyl-1,2,3,4-tetrahydro-cyclopenta[b]indol-2-yl)-carbamic acid isopropyl ester (TT701) to treat androgen deprivation therapy associated symptoms. Data was presented therein from various animals including rats and dogs that showed treatment with TT701, at the doses provided, for a period of 1 to 12 months, decreased prostate size in the rats and dogs which indicated that the compound does not accrue androgenic risk of prostate hyperplasia over time. The treatment of TT701 in dogs for 6 and 12 months resulted in a 60% to 80% decrease in prostate weight and the presence of atrophy. This data alone, or the other safety or clinical data disclosed therein, is not dispositive of the treatment of any indication in humans except for the treatment of androgen deprivation symptoms. The data shown therein also disclosed that there were no significant changes from baseline in prostate specific antigen (PSA) levels when compared with placebo at any time point or any dose tested of TT701 in healthy volunteers. The present invention broadly relates to the discovery that a compound of formula I, inclusive of TT701, and, optionally, a combination with a PDE-5 inhibitor is (are) useful for the treatment of the signs and symptoms of BPH in patients in need of treatment thereof. The term “patient” or “patients” is inclusive of humans and animals.

The combination of pre-clinical data and clinical data in healthy patients provided data that was supportive of the prior disclosed uses of TT701. New data generated in patients having erectile dysfunction treated with OPK88004 (TT701) alone, or in combination with tadalafil, surprisingly and unexpectedly showed a significant reduction in PSA levels in those patients having PSA levels of ≥2.0 ng·ml. This is also in sharp contrast to the data shown with respect to healthy volunteers. The present invention is thus a method of treating patients having the signs and symptoms of BPH by reducing PSA by at least 5, 10, 15 or 20% and reducing prostate size and volume.

### BRIEF DESCRIPTION OF THE FIGURES

FIG. 1 illustrates the results that daily oral administration of the compound of Formula II led to a dose-dependent increase in vertebral bone mineral content (BMC), cross-sectional area, and bone mineral density (BMD).

FIG. 2 illustrates the results of one way analysis of levator ani W/BW (mg/g) in delayed rat ORX model.

FIG. 3 illustrates the results of the compound of Formula II treatment in the ORX rat that showed minimal accrual SV/Prostate risk

FIG. 4 illustrates the effect of Testosterone Enanthate (TE) and the compound of Formula II on Prostate weight.

FIG. 5 illustrates the effect of Testosterone Enanthate (TE) and the compound of Formula II on Seminal Vesicle weight.

FIG. 6 illustrates the results of the compound of Formula II Phase I PSA results in healthy volunteers (ug/L).

FIG. 7 illustrates the PSA mean change from baseline by treatment and visit day in the healthy volunteers: Study GPEC (nanogram/mL).

FIGS. 8 and 9 show the changes in PSA levels (ng/ml) in patients having symptoms of erectile dysfunction and who were failing tadalafil after treatment using OPK-88004 (TT701) alone or in combination with tadalafil (5 mgs and 5 mgs). FIG. 8 also shows the data with respect to tadalafil alone.

FIG. 10 shows the changes in PSA levels in patients having symptoms of erectile dysfunction and who were failing tadalafil treatment being administered OPK-88004 (TT701) alone or in combination with tadalafil (5 mgs alone or 5 mg/5 mg combination) where the PSA horizontal scale is broadened. This figure shows a decrease in PSA levels at about 2.0 ng ml upon treatment with OPK-88004.

FIG. 11 illustrates that the compound of Formula II causes no change in Hematocrit with TT701 in 12 weeks.

FIG. 12 illustrates the LBM mean change from baseline to week 12: Study GPEC.

FIG. 13 illustrates the Muscle Power (stair climb) mean change from baseline to week 12: Study GPEC.

FIG. 14 illustrates the fat Mass mean change from baseline to week 12: Study GPEC.

FIG. 15 shows the study design outlined in Example 15.

FIG. 16 shows the changes in PSA levels in patients being administered tadalafil alone (5 mg and 10 mg) or being administered OPK-88004 (TT701) (1 mg or 5 mg) in combination with tadalafil (5 mg) after 12 weeks of treatment.

### DETAILED DESCRIPTION OF THE INVENTION

The invention encompasses methods of treating the signs and symptoms of BPH by administering at least a compound of Formula I in a therapeutically effective amount to a subject in need of treatment thereof. Also, the invention encompasses treating the signs and symptoms of BPH by administering a pharmaceutical composition comprising at least one compound of Formula I or a pharmaceutically acceptable salt thereof and a pharmaceutically acceptable excipient and dosage forms thereof. The compounds of Formula I may be prepared by the methods described in U.S. Pat. No. 7,968,587, hereby incorporated by reference or as described in WO 2016/040234 A1 (PCTUS2015/048801) which is hereby incorporated by reference.

The inventors found that compounds for Formula I and, in particular, a compound of Formula II (described below, also known as LY2452473, TT701, OPK-88004 or (S)-(7-cyano-4-pyridin-2-ylmethyl-1,2,3,4-tetrahydro-cyclopenta[b]incol-2-yl)-carbamic acid isopropyl ester), are potent and selective modulators of the human androgen receptor (hAR) in cell based assays. Tests with prostate cancer cell lines found that a compound of Formula II was at least 46 fold weaker in inducing gene expression than synthetic testosterone, and yet the binding affinity was only four fold lower. This difference between binding affinity and gene expression is significant and indicates that binding to the prostate androgen receptors provides only weak agonist activity and at much higher concentrations. The compounds of the invention (those of Formula I and in particular Formula II) are unique in that they reduce prostate volume at low doses (3 mg/kg) and also at very high doses (300 mg/kg). The compounds are potent antagonists on the prostate at low doses and furthermore lack agonist activity at very high doses. Because of the interaction of a compound of formula II with the androgenic receptor in the prostate, these data suggest that such compound of formula II is a very potent antagonist to the prostate androgenic receptor at low doses. Further, one would have expected that high doses of this compound would activate these receptors and stimulate prostate hypertrophy. Surprisingly, this did not occur. More importantly, the combination of these earlier findings coupled with the new clinical data regarding the effect of treatment with OPK-88004 on PSA levels in patients having erectile dysfunction and failing on tadalafil therapy provides clinical basis to support a method of treating the signs and symptoms of BPH in patients in need of treatment thereof. These unexpected results obtained with the compounds of the invention make them unique for treating the signs and symptoms of BPH, this is particularly true for the compound of Formula II.

As used herein the term “(C₁-C₄)alkyl” means a straight or branched, monovalent, saturated aliphatic chain of one to four carbon atoms.

As used herein, the term (C₁-C₄)alkoxy means an oxygen atom bearing a straight or branched alkyl chain as described above.

As used herein, the terms “halo,” “halide,” or “Hal” refer to chlorine, bromine, iodine or fluorine unless stated otherwise.

As used herein, the term “patient” includes mammals such as humans, dogs, cats, cows, horse, pigs, or sheep or other mammal.

As used herein, the term “treating” or “treatment” means administering at least one drug or a combination thereof to alleviate and treat the underlying signs, causes or symptoms of a disease or condition. This term includes any form of prohibiting, slowing, stopping or otherwise interfering with disease progression. The preferred mammal to treat is humans and the indication being treated is benign prostatic hyperplasia (BPH). The preferred patient population is those patients having BPH and having a PSA level of greater than about 2.0. The most preferred population has a PSA level of greater than about 2.5. This disease or condition is or involves the presence of an enlarged prostate gland. The symptoms of this condition involves squeezing or partial blockage of the urethra.

As used herein, the terms “T1-T4” refer to the T category of the TNM staging system of the American Joint Committee on Cancer (AJCC) to describe how far a cancer has spread. The T category indicates the presence of tumors and describes the extent of the primary tumor. Higher numbers indicate increased size, extent, or degree of penetration. Each cancer type has specifics to classify under the number. For prostate cancer, T1 indicates that the doctor cannot feel the tumor or see it with imaging such as transrectal ultrasound. T2 indicates that the doctor can feel the cancer with a digital rectal exam (DRE) or see it with imaging such as transrectal ultrasound, but it still appears to be confined to the prostate gland. T3 indicates that the cancer has begun to grow and spread outside the prostate and may have spread into the seminal vesicles. T4 indicates that the cancer has grown into tissues next to the prostate (other than the seminal vesicles), such as the urethral sphincter (muscle that helps control urination), the rectum, the bladder, and/or the wall of the pelvis.

As used herein, the term “effective amount” refers to the amount or does of compound of Formula I, or a pharmaceutically acceptable salt thereof, upon administration to the patient, provides the desired effect in the patient under diagnosis or treatment. In determining the effective amount for a patient, a number of factors are considered by the attending diagnostician including, but not limited to, the patient's size, age, and general health; the specific disease or disorder; the response of the individual patient; the particular compound administered; the mode of administration; the bioavailability characteristics of medication; and other relevant circumstances.

Clinical trials measuring the efficacy or potential efficacy of a compound of Formula I or a combination of a compound of Formula I with a second active ingredient (such as PDE-5 inhibitors) are conducted by measuring, as a primary endpoint, the International Prostate Symptom Score (IPSS). The IPSS is a four week recall questionnaire administered to both a placebo group and after randomization. The IPSS is used to assess the severity of irritative symptoms such as frequency, urgency or straining as well as obstructive symptoms such as incomplete emptying, stopping and starting, weak stream and straining or pushing. The IPSS' numeric scores can range from 0 to 35 where higher scores are indicative of a more severe condition. A secondary endpoint in clinical studies conducted for BPH includes measuring the maximum urinary flow rate (Qmax). Clinical trials are conducted in patients having BPH using two dosage strengths of a compound of formula II (3 mgs and 5 mgs once a day) over a 24 month period relative to placebo. In addition, clinical trials are conducted on a fixed combination dose of 5 mgs of a compound of formula II and 5 mgs of tadalafil relative to placebo over a 24 month period. These studies are similar to other clinical trials conducted to assess the efficacy of compounds to treat BPH.

In clinical studies conducted for the approval of CIALIS® (tadalafil, 5 mgs/day), a total of 748 patients (N=748) were enrolled in either placebo or drug trials to measure the above primary and secondary endpoints for the treatment of patients having both symptoms of BPH, or erectile dysfunction (ED) and BPH. As stated above, the clinical trials to assess the activity of compounds of Formula I with or without a second active ingredient are conducted in a similar manner to those described for the clinical studies supporting the approval of CIALIS® (tadalafil) for the treatment of BPH.

Animal studies determined that the compound of Formula II (TT701) showed selectivity for the anabolic effects relative to the prostate androgenic effects. The ED₅₀ for the levator ani muscle was 1-3 mg/kg whereas doses of 30 mg/kg, the highest dose examined, did not induce changes in the prostate of orchidectomized rats. This result suggests at least a 10-30 fold selectivity.

When studying BPH, the treatment of normal dogs with the compound of Formula II yielded a progressive decrease in prostate size by 60% over a six-month treatment period. Similar antagonist effects on prostate weight were observed with treatment doses of 3, 30 and 300 mgs/kg, whereas increase in anabolic activity was observed in skeletal muscle and bone. These data support that compounds of Formula I and, in particular, Formula II work as antagonists to endogenous androgenic related effects on the prostate.

Orchidectomized rats with reduced prostate weights were treated with testosterone alone or with testosterone and the compound of Formula II. Testosterone treatment alone only partially reversed the effect, however it also increased prostate weight. In contrast, the combination of testosterone and the compound of Formula II reduced testosterone induced effects on prostate size, indicating that the compound of Formula II may act as an androgen antagonist on the prostate.

In clinical studies in patients with androgen deficiency, treatment with TT701, the compound of Formula II, resulted in a 20-30% decrease in the levels of endogenous testosterone. The exact effect of this decrease in testosterone levels on androgen related anabolic and prostate effects is not known, but may be dependent on the base levels of testosterone.

In these same clinical studies with in patients with androgen deficiency, TT701 demonstrated clinically and statistically significant increases in lean body mass and changes in bone biochemical biomarkers consistent with a bone anabolic increase. No increases in PSA were observed at any dose level (up to 75 mg doses) indicating that the compound of Formula II acts as a selective AR modulator in humans (agonist effects on some tissues, neutral or antagonistic effect on the prostate), supporting the data generated in animal models.

In clinical studies for the potential treatment of patients with androgen deficiency, TT701 showed a good safety profile within the dose ranges studied. The major changes observed in patients treated with 5 mg of the compound of Formula II for 12 weeks was a 20% decrease in HDL, and some decrease in sex hormone binding globulin, LH and FSH, but the magnitude of these findings were not considered clinically relevant.

The present invention also relates to use of TT701 and compounds of formula I in the treatment of the signs and symptoms of BPH and the symptoms of androgen deficiency in men. These symptoms include sexual symptoms, fatigue, low vitality and physical dysfunction. The combination of a compound of formula II herein and tadalafil in a single dosage form is a particular preferred treatment for BPH and any of the symptoms delineated above.

The compound of Formula II acts as a SARM in humans with an agonist effect on some tissues while sparing the prostate or potentially antagonizing androgen related effects on the prostate. These data indicate that the compound of Formula II reduces prostate size and increases the pelvic floor muscles. Optionally, the compounds of Formula I and Formula II may be administered as single agents or as combinations with additional drugs, such as PDE-5 inhibitors, to treat BPH. The combination may not only slow the progression of BPH, but also reduce the urinary tract symptoms and obstruction. Animal and human safety data indicated that the compound of Formula II has an acceptable safety profile

Phosphodiesterase type-5 (PDE-5) inhibitors include, but are not limited to, sildenafil, vardenafil, or tadalafil. The latter active ingredient has been approved for both erectile dysfunction and the signs and symptoms of BPH. Certain drugs have been co-administered in separate dosage forms in clinical studies for the treatment of erectile dysfunction, including the co-administration of tadalafil and the compound of Formula II at particular strengths. However, there is no disclosure of a method of treating the signs and symptoms of BPH in a co-administered regimen. There is a need for additional compounds and combinations thereof that are suitable for the treatment of BPH.

The present invention comprises a method of treating the signs and symptoms of BPH by administering at least one compound of Formula I in a therapeutically effective amount to a subject in need thereof. The present invention also encompasses treating the signs and symptoms of BPH by administering at least one compound of Formula I in combination with at least one phosphodiesterase type-5 (PDE-5) inhibitor. When administered as a combination, the combination includes simultaneous or sequential administration of a single dosage or separate dosages form. For instance, when administered as separate dosage form, a first dosage form comprises a compound of Formula I and a second dosage form comprises a PDE-5 inhibitor. Simultaneous administration may include a single dosage form wherein a first active ingredient selected from a compound of Formula I and a second active ingredient selected from a PDE-5 inhibitor are provided in a single dosage form. Alternatively, simultaneous administration may include two separate dosage forms, a first dosage form with an active ingredient selected from a compound of Formula I and a second dosage form with an active ingredient selected from a PDE-5 inhibitor are provided as two separate dosage forms taken at once or sequentially as prescribed.

The present invention also relates to a method of treating the signs and symptoms of BPH in patients in need of treatment thereof, comprising administering to the patient an effective amount of a compound of formula I or a pharmaceutically acceptable salt thereof sufficient to reduce the patient's prostate specific antigen (PSA) levels by at least 5, 10, 15, 20 or 25% while efficaciously reducing prostate size or volume.

In a preferred embodiment, the present invention comprises a method of treating the signs and symptoms of BPH patients having a PSA level of about 2.0 or greater with a pharmaceutically effective amount of a compound of formula I or a salt thereof and wherein said effective amount is sufficient to lower PSA levels by at least 5, 10, 15 or 20% while reducing prostate size or volume.

In a preferred embodiment, the present invention comprises a method of treating the signs and symptoms of BPH patients having a PSA level (ng/ml) of about 2.1, 2.2, 2.3, 2.4 or greater with a pharmaceutically effective amount of a compound of formula I or a salt thereof and wherein said effective amount is sufficient to lower PSA levels by at least 5, 10, 15, 20% or greater while reducing prostate size or volume. The invention also relates to a method of treating the signs and symptoms of BPH and reducing PSA levels in a patient in need of treatment thereof comprising administering a pharmaceutically effective amount of a compound of formula I and, optionally, a PDE-5 inhibitor in patients having a PSA level of between 2.5-4.5 ng/ml and wherein the % reduction in said PSA levels ranges from 10 to 30%.

The present invention relates to a method of treating the signs and symptoms of BPH in patients having a need of treatment thereof comprising administering to the patient an effective amount of an oral dosage form comprising a compound of formula I or a pharmaceutically acceptable salt thereof sufficient to reduce the patient's PSA levels and to reduce prostate size or volume.

The present invention also relates to a method of treating the signs and symptoms of BPH in a patient in need of treatment thereof wherein a dosage form comprising a compound of formula I or pharmaceutically acceptable salts or enantiomers or polymorphs thereof is effective to reduce the patient's PSA levels to a range of 0 to 6.5 ng/mL.

The invention also comprises a method of treating a patient having BPH comprising administering a pharmaceutically effective amount of a compound of formula 1 or a pharmaceutically acceptable salt thereof in a once-a-day dosage form and wherein said patient's PSA levels are reduced by at least 10 to 25% relative to pre-treatment levels.

The invention comprises a combination of a PDE-5 inhibitor and a compound of formula I or pharmaceutically acceptable salts thereof wherein said combination is effective in a method to reduce PSA and treat BPH in patients in need of treatment thereof.

The invention further relates to a dosage form comprising a compound of formula I and, optionally, a PED-5 inhibitor for use in the treatment of the signs and symptoms of BPH wherein the compound of formula I has at least one of the data points described in Table 1 below as well as a range of 30% on each side of each data point selected from the group consisting of AR Ki (nM); AR C2C12 EC50 (nM), ER, GR, PR, MR (IC50/Ki)(nM), muscle LA ED (mg/kg), bone Eff (BM) (mgs/kg), Rat uterine risk (mgs/kg), Rat SV/Prost risk (mgs/kg), Rat F %, Dog F % and MOS (AUC) and wherein said compound is effective to lower PSA and reduce prostate size or volume.

TABLE 1

| | |
|---|---|
| Assay | TT701 |
| AR Ki (nM) | 1.95 +/− 30% |
| AR C2C12 | 1.25 +/− 30% |
| EC50 (nM) | |
| ER, GR, PR | >3,000 +/− 30% |
| MR(IC50/Ki)(nM) | |
| Muscle LA ED | 3 mpk +/− 30% |
| mg/kg | |
| Bone Eff (BM) | 2 mpk +/− 30% |
| Rat uterine risk | 20 mpk +/− 30% |
| Rat SV/Prost risk mgs/kg | >30 mpk +/− 30% |
| Rat F % | 63 +/− 30% |
| Dog F % | 58 +/− 30% |
| MOS (AUC) | 28X_{dog} + |
| | 71X_{cat} +/− 30% |

The claimed invention further relates to a method of prolonging the duration of action of a PDE-5 inhibitor in the treatment of the signs and symptoms of BPH in patients in need of treatment thereof by administering a pharmaceutically effective amount of a compound of formula I in combination with said PDE-5 inhibitor to treat BPH in said patient for a period of at least one to twenty-four months or longer beyond the duration of action of said PDE-5 inhibitor administered alone.

The present invention further relates to a method of treating the signs and symptoms of BPH in a patient in need of treatment thereof with a fixed combination dosage form wherein the combination comprises a first compound selected from a compound of formula I and a second compound selected from a PDE-5 inhibitor and wherein (1) the duration of action in the treatment of the signs and symptoms of BPH is extended beyond the duration for the second compound alone and (2) the patient has a PSA level of greater than 2.5.

The present invention further relates to a method of extending the treatment period for a PDE-5 inhibitor in the treatment of the signs and symptoms of BPH comprising co-administering a combination of a selective estrogen receptor modulator (SARM) and said PDE-5 inhibitor. The SARM may be selected from any SARM known in the art and any PDE-5 inhibitor. The combination may also be a fixed-combination dosage form.

The dosage amounts or strengths of each active ingredient alone or in the combination form are selected and prescribed by a physician. Such dosages for the SARM include those doses and strengths generally described in U.S. Pat. No. 7,968,587 for the treatment of androgen disorders excluding the contribution of the dosage strength from the second active ingredient (such as, a PDE-5 inhibitor), which may be prescribed within those ranges known to treat erectile dysfunction and BPH. It is believed that the combination of active ingredient 1 (SARM) with active ingredient 2 (PDE-5 inhibitor) leads to at least an additive effect with a prolonged duration of action and/or a synergistic effect that either (1) maintains the relative efficacy of any given amount of each active ingredient were it to be administered alone to treat BPH and/or (2) increases the duration of action to treat BPH due to the particular properties of the SARM(s) utilized herein.

Thus, it is also believed that the action of such a combination will be useful to extend, for example, the duration of efficacious treatment of the signs and symptoms of BPH in a patient receiving such a combination dosage form relative to a patient receiving tadalafil alone for such treatment. There is at least anecdotal evidence that other drugs used to treat BPH lose their effectiveness over time as the disease progresses and as the prostate continues to grow. The present invention thus relates to a method of increasing the efficacy over a six to 24 or longer month period comprising administering a pharmaceutically effective amount of a compound of formula I to a patient in need of treatment thereof.

The present invention comprises a method of treating the signs and symptoms of BPH in a patient in need of treatment thereof comprising administering a compound of formula I or a pharmaceutically effective salt thereof and, optionally, a PDE-5 inhibitor to said patient wherein the efficacy of such compound in treating the signs and symptoms of BPH or a symptom thereof is for a period of at least 6, 10, 12, 14, 16, 18, 20, 22 or 24 months or longer.

The compounds of the first active ingredient as described herein are those of Formula I:

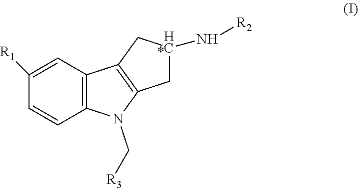

wherein the C* atom may be R, S or R/S configuration;

R₁ represents cyano, —CH═NOCH₃, —OCHF₂, or —OCF₃;

R₂ represents —COR₂ₐ or —SO2R_{2b};

R₂ₐ represents (C₁-C₄)alkyl, (C₁-C₄)alkoxy, cyclopropyl, or —NRaRb;

R_{2b} represents (C₁-C₄)alkyl, cyclopropyl, or —NRaRb;

Ra and Rb each independently is H or (C₁-C₄)alkyl; and

R₃ represents a heteroaryl group selected from the group consisting of pyridinyl, pyrimidinyl, pyrazinyl, pyridazinyl, thiazoloy isothiazolyl, and thiadiazolyl, each optionally substituted with 1 or 2 substituents independently selected from the group consisting of methyl, ethyl, bromo, chloro, fluoro, —CHF₂, —CF₃, hydroxyl, amino and —NHCH₂CO₂H, or a pharmaceutically acceptable salt thereof.

Preferred compounds of the invention include those wherein R₂ and R₃ are any of the variables as defined herein and:

R₁ is CN, —CH═NOCH₃ or —OCF₃ or;

R₁ is CN or —CH═NOCH₃; or

R₁ is CN or

R₁ is —CH═NOCH₃.

In another preferred set of compounds of Formula I, R₁ and R₃ have any of the variables as defined herein and:

R₂ is —COR₂ₐ or —SO2R_{2b} wherein R₂ₐ is (C₁-C₄)alkyl, (C₁-C₄)alkoxy, cyclopropyl, or —N(CH₃)₂ and R_{2b} is (C₁-C₄)alkyl, cyclopropyl, —N(CH₃)₂ or —N(C₂H₅)₂; or

R₂ is —COR₂ₐ or —SO₂R_{2b} wherein R₂ₐ is ethyl, isopropyl, methoxy, ethoxy, propoxy, isopropoxy, isobutoxy, tert-butoxy, cyclopropyl, or —N(CH₃)₂ and R_{2b} is methyl, ethyl, propyl, cyclopropyl, —N(CH₃)₂ or —N(C₂H₅)₂; or

R₂ is —COR₂ₐ wherein R₂ₐ is selected from ethyl, isopropyl, methoxy, ethoxy, propoxy, isopropoxy, isobutoxy, tert-butoxy, cyclopropyl, or —N(CH₃)₂; or

R₂ is —COR₂ₐ, wherein R₂ₐ is isopropyl, ethoxy, isopropoxy or cyclopropyl; or

R₂ is —COR₂ₐ wherein R₂ₐ is isopropoxy; or

R₂ is —SO₂R_{2b}, wherein R_{2b} is methyl, ethyl, propyl, cyclopropyl, —N(CH₃)₂ or —N(C₂H₅)₂; or

R₂ is —SO₂R_{2b} wherein R_{2b} is cyclopropyl or —N(CH₃)₂; or

R₂ is —SO₂R_{2b} wherein R_{2b} is —N(CH₃)₂.

Another preferred set of compounds of Formula I include those wherein R₁ and R₃ have any of the values as recited herein and R₂ is —COR₂ₐ and the “C*” carbon center is in the S configuration; or R₂ is —SO₂R_{2b} and the “C*” carbon center is in the R configuration.

Other preferred compounds used for the treatment of the signs and symptoms of BPH include those compounds of Formula I wherein R₁ and R₂ have any of the values recited herein and

R₃ is a heteroaryl group selected from the group consisting of pyridinyl, pyrimidinyl, pyrazinyl, pyridazinyl, thiazolyl, isothiazolyl, and thiadiazolyl, each optionally substituted with one or more substituents independently selected from the group consisting of methyl, bromo, chloro, fluoro, —CHF₂, hydroxyl, amino and —NHCH₂CH₂CO₂H; or

R₃ represents 6-fluoro-pyridin-2-yl, pyridine-2-yl, 3-hydroxy-pyridin-2-yl, 6-difluoromethyl-pyridin-2-yl, 2-amino-pyridin-3-yl, 2-carboxymethylamino-pyridin-3-yl, pyrimidin-4-yl, pyrimindin-2-yl, 2-chloro-pyrimidin-4-yl, thiazol-4-yl, 2-methyl-thiazol-4-yl, 2-chloro-thiazol-4-yl, thiazol-2-yl, thiazol-5-yl, thiazol-5-yl, 4-amino-thiazol-5-yl, pyrazine-2-yl, 5-methyl-pyrazin-2-yl, 3-chloro-pyrazin-2-yl, pyridazin-3-yl, 5-bromo-isothiazol-3-yl, isothiazol-3-yl, 4,5-dichloro-isothiazol-3-yl, or [1,2,5]thiadiazol-3-yl; or

R₃ is selected from 6-fluoro-pyridin-2-yl, pyridine-2-yl, 3-hydroxy-pyridin-2-yl, 6-difluoromethyl-pyridin-2-yl, 2-amino-pyridin-2-yl, 2-carboxymethylamini-pyridin-3-yl, thiazol-4-yl, 2-methyl-thiazol-4-yl, 2-chloro-thiazol-4-yl, thiazol-2-yl, thiazol-5-yl, 4-amino-thiazol-5-yl, pyrazine-2-yl, 5-methyl-pyrazin-2-yl, 3-chloropyrazin-2-yl, 6-methyl-pyrazin-2-yl, 3-amino-pyrazin-2-yl or 3-methyl-pyrazin-2-yl; or

R₃ is selected from 6-fluoro-pyridin-2-yl, pyridine-2-yl, 2-amino-pyridin-3-yl, thiazol-5-yl or 4-amino-thiazol-5-yl; or

R₃ is selected from pyridine-2-yl, 2-amino-pyridin-3-yl, thiazol-5-yl or 4-amino-thiazol-5-yl.

Another set of preferred compounds used to treat BPH includes compounds of formula I wherein when R₂ is —COR₂ₐ, the “C*” carbon is in the S configuration; and when R₂ is —SO₂R_{2b}, the “C*” carbon is in the R configuration; R₁ is selected from cyano or —CH═NOCH₃; R₂ is selected from —COR₂ₐ or —SO₂R_{2b} wherein R₂ₐ represents (C₁-C₄)alkyl-, (C¹-C₄)alkoxy-, cyclopropyl, or —N(CH₃)₂ and R_{2b} represents (C₁-C₄)alkyl, cyclopropyl, —N(CH₃)₂ or —N(C₂H₅)₂; and R₃ represents a heteroaryl group selected from the group consisting of pyridinyl, pyrimidinyl, pyrazinyl, pyridazinyl, thiazolyl, isothiazolyl, and thiadiazolyl, each of which is independently selected from the group consisting of methyl, bromo, chloro, fluoro, —CHF₂, hydroxyl, amino, and —NHCH₂CO₂H.

A particularly preferred compound used for treating the signs and symptoms of BPH is represented by Formula (I)a:

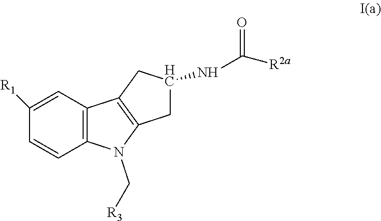

wherein,

R₁ is cyano, —CH═NOCH₃, or —OCF₃;
R^{2a} is —(C₁-C₄)alkyl, (C₁-C₄)alkoxy-, cyclopropyl or —N(CH₃)₂; and
R₃ represents a heteroaryl group selected from the group consisting of pyridinyl, pyrimidinyl, pyrazinyl, pyridazinyl, thiazolyl, isothiazolyl, and thiadiazolyl, optionally substituted with at least one of methyl, bromo, chloro, fluoro, —CHF₂, hydroxyl, amino, or —NHCH₂CO₂H.

Even more preferred compounds for treating the signs and symptoms of BPH are compounds of formula I(a) wherein R₁ is cyano or —CHNOCH₃; R₂ₐ is selected from the group consisting of ethyl, isopropyl, methoxy, ethoxy, propoxy, isopropoxy, isobutoxy, tert-butoxy, cyclopropyl, or —N(CH₃)₂; and R₃ is selected from the group consisting of pyridin-2-yl, 2-amino-pyridin-3-yl, thiazol-5-yl, or 4-amino-thiazol-5-yl. The most preferred compound used in the method of the invention is a compound of formula II and pharmaceutically acceptable salts thereof:

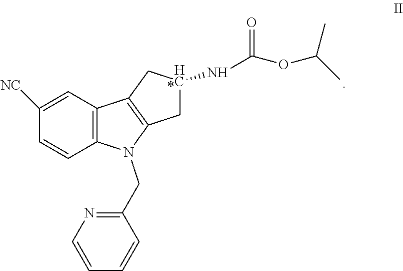

In one embodiment, the administration of compounds of Formula I or Formula II, alone or in combination with a phosphodiesterase type-5 inhibitor, improve lower urinary tract symptoms (LUTS) associated with BPH in a subject. In another embodiment, compounds of Formula I or Formula II, alone or in combination with a phosphodiesterase type-5 inhibitor, are administered to subjects having moderate-to-severe BPH-LUTS. In another embodiment, compounds of Formula I or Formula II, alone or in combination with a phosphodiesterase type-5 inhibitor, are administered to subjects having an enlarged prostate. In another embodiment, the administration of compounds of Formula I or Formula II, alone or in combination with a phosphodiesterase type-5 inhibitor, reduce the risk of urinary retention in a subject by affecting the excessive growth of the prostate. In another embodiment, the administration of compounds of Formula I or Formula II, alone or in combination with a phosphodiesterase type-5 inhibitor, increases lean body mass (LBM) and calf area in a subject. In another embodiment, the administration of compounds of Formula I or Formula II, alone or in combination with a phosphodiesterase type-5 inhibitor, decreases fat mass in a subject. In another embodiment, the administration of compounds of Formula I or Formula II, alone or in combination with a phosphodiesterase type-5 inhibitor, increases lower extremity muscle power in a subject. In another embodiment, the administration of compounds of Formula I or Formula II, alone or in combination with a phosphodiesterase type-5 inhibitor, improves physical function or fatigue in a subject. In another embodiment, the administration of compounds of Formula I or Formula II, alone or in combination with a phosphodiesterase type-5 inhibitor, provides positive anabolic effects on the maintenance of muscle mass and strength.

In one embodiment, the subject selected for administration of compounds of Formula I or Formula II, alone or in combination with a phosphodiesterase type-5 inhibitor, is at least 40 years old. In another embodiment, the subject selected for administration of compounds of Formula I or Formula II, alone or in combination with a phosphodiesterase type-5 inhibitor, is at least 50 years old.

In one embodiment, the subject selected for administration of compounds of Formula I or Formula II, alone or in combination with a phosphodiesterase type-5 inhibitor, is identified by having an enlarged prostate.

The compounds of the invention are made by alkylating a tetrahydrocyclopenta[b]indole compound with the appropriate alkylating agent of the formula R₃—CH₂—X wherein X is a leaving group (halogen) and R₃ is defined as recited herein. U.S. Pat. No. 7,968,587 which describes the synthesis of such compounds and is hereby incorporated by reference.

Compounds of the present invention may be formulated as part of a pharmaceutical composition. As such, a pharmaceutical composition comprising a compound of Formula (I), or a pharmaceutically acceptable salt thereof, in combination with a pharmaceutically acceptable carrier, diluent or excipient is an important embodiment of the invention. Examples of pharmaceutical compositions and methods for their preparation are well known in the art. See, e.g. REMINGTON: THE SCIENCE AND PRACTICE OF PHARMACY (A. Gennaro, et al., eds., 19.sup.th ed., Mack Publishing (1995)). Illustrative compositions comprising compounds of Formula (I) include, but are not limited to, a compound of Formula (I) in suspension with 1% sodium carboxymethyl cellulose, 0.25% polysorbate 80, and 0.05% Antifoam 1510™ (Dow Corning); or a compound of Formula (I) in suspension with 0.5% methylcellulose, 0.5% sodium lauryl sulfate, and 0.1% Antifoam 1510 in 0.01N HCl (final pH about 2.5-3).

The invention also provides pharmaceutical compositions comprising one or more compounds of Formula I in association with a pharmaceutically acceptable carrier. Preferably these compositions are in unit dosage forms such as tablets, pills, capsules, powders, granules, sterile parenteral solutions or suspensions, metered aerosol or liquid sprays, drops, ampoules, auto-injector devices or suppositories; for oral, parenteral, intranasal, sublingual or rectal administration, or for administration by inhalation or insufflation. It is also envisioned that the compounds of the present invention may be incorporated into transdermal patches designed to deliver the appropriate amount of the drug in a continuous fashion. The preferred dosage form is an oral capsule or tablet. A compound of Formula (I), or a composition comprising a compound of Formula (I) can be administered by any route which makes the compound bioavailable, including oral and parenteral routes.

Clinical studies have demonstrated that up to 1000 mgs/day of TT701 is safe. A dosage range for a compound of Formula I or II is between 1 mg to about 1,000 mg per day. For example, dosages per day of individual agents normally fall within the range of about 1 mg/day to about 1000 mg/day; about 1 mg/day to about 500 mg/day; about 1 mg/day to about 250 mg/day; about 1 mg/day to about 100 mg/day; about 1 mg/day to about 75 mg/day; and about 1 mg/day to about 25 mg/day. Other dosages per day of individual agents normally fall within the range of 1 mg/day to about 5 mg/day. Typically, the compound of Formula I is used at a dose per day selected from 1 mg, 5 mg, 25 mg, or 75 mg per day.

A preferred dosage range for compounds of Formula I or Formula II is about 0.5 mg to about 50 mg. A more preferred dosage is about 1 mg to about 5 mg. Optionally the doses can be administered with 5 mg of tadalafil. For example, a dose may include 5 mg of the compound of Formula I and 5 mg of tadalafil. Alternatively, the dose may be in terms of mg/kg. In this format, a typical dose is about 0.02 mg/kg to about 0.1 mg/kg. For example, most patients are adult men who are 50 to 120 kg so a narrow mg/kg range might be from 0.02 mg/kg (1 mg to 50 kg patient) to 0.1 mg/kg (10 mg to 100 kg patient).

In one embodiment, the compounds of Formula I or Formula II are administered to a subject at a dosage of 5 mg, 15 mg, or 25 mg once daily.

In another embodiment the compounds of Formula I is administered to a subject in a dose ranging from 0.0001 to 5 mg per day. In another embodiment the compounds of Formula I is administered to a subject in a dose ranging from 5 to 15 mg per day. In another embodiment the compounds of Formula I is administered to a subject in a dose ranging from 15 to 25 mg per day.

In another embodiment the compounds of Formula II is administered to a subject in a dose ranging from 0.0001 to 5 mg per day. In another embodiment the compounds of Formula II is administered to a subject in a dose ranging from 5 to 15 mg per day. In another embodiment the compounds of Formula II is administered to a subject in a dose ranging from 15 to 25 mg per day.

In one embodiment, the compounds of Formula I or Formula II are administered once daily for a period of at least four weeks. In another embodiment, the compounds of Formula I or Formula II are administered once daily for a period of at least eight weeks. In another embodiment, the compounds of Formula I or Formula II are administered once daily for a period of at least twelve weeks. In another embodiment, the compounds of Formula I or Formula II are administered once daily for a period of at least sixteen weeks. In another embodiment, the compounds of Formula I or Formula II are administered once daily for a period of at least twenty weeks. In another embodiment, the compounds of Formula I or Formula II are administered once daily for a period of up to six months. In another embodiment, the compounds of Formula I or Formula II are administered once daily for a period of up to two years.

For preparing solid compositions such as tablets, the principal active ingredient (the compound of Formula I) is mixed with a pharmaceutically acceptable carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture for a compound of the present invention, or a pharmaceutically acceptable salt thereof. When referring to these preformulation compositions as homogeneous, it is meant that the active ingredient is dispersed evenly throughout the composition so that the composition may be easily subdivided into equally effective unit dosage forms such as tablets, pills and capsules. This solid pre-formulation composition is then subdivided into unit dosage forms of the type described above containing from 0.1 to about 500 mg of the active ingredient. Typical unit dosage forms contain from 1 to 100 mg, for example, 1, 2, 5, 10, 25, 50 or 100 mg, of the active ingredient. The tablets or pills of the composition can be coated or otherwise compounded to provide a dosage affording the advantage of prolonged action. For example, the tablet or pill can comprise an inner dosage and an outer dosage component, the latter being in the form of an envelope over the former. The two components can be separated by an enteric layer which, serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinylpyrrolidone or gelatin.

In one embodiment, the compounds of Formula I or Formula II are administered orally in a gelatin capsule. In another embodiment, each gelatin capsule for oral administration contains the compounds of Formula I or Formula II, inactive ingredients, pregelatinized starch and dimethicone. In one embodiment, the gelatin capsules containing at least the compounds of Formula I or Formula II have at least one of the data points described in Table 2 below as well as a range of 30% on each side of each data point selected from the group consisting of the following properties: assay, un-specified impurity, total impurities, water activity, dissolution.

TABLE 2

| Test | Acceptance Criteria | Result |
|---|---|---|
| Physical | Blue Capsules | Pass |
| Appearance | | |
| (Visual) | | |
| Package | Report Results | Pass |
| Characteristics | | |
| (Visual) | | |
| Assay | NLT 90.0% and NMT | 100.0% to 101.6% |
| (HPLC) | 110.0% of the label | |
| | claim | |
| Any Unspecified | Impurities: Individual | 0.49% to 0.58% |
| Impurity | Percent | |
| (HPLC) | | |
| Total Impurities | Impurities: Total | 0.49% to 0.58% |
| (HPLC) | Percent | |
| Water Activity | Report results | 0.29 aw to 0.32 aw |
| Dissolution | Meets USP <711> with | Pass |
| (Apparatus | Q = 75% at 45 minutes | Low = 99 to 101% |
| II/HPLC) | (No. of Units = 6) | High = 102 to 104% |
| | | Avg. = 101 to 102% |

In one embodiment, the gelatin capsules containing at least the compounds of Formula I or Formula II has a potency of at least 90.0% when measured using an assay. In another embodiment, the gelatin capsules containing at least the compounds of Formula I or Formula II has a potency of not more than 110.0% when measured using an assay. In another embodiment, the gelatin capsules containing at least the compounds of Formula I or Formula II meets the requirements set forth in <905> of the United States Pharmacopeial Convention. In another embodiment, the gelatin capsules containing at least the compounds of Formula I or Formula II has the following microbial limits: TMAC<1000 cfu/g, TYMC<100 cfu/g; Absence of Escherichia coli/1 g.

The compounds of the present invention are useful when formulated in the form of a pharmaceutical injectable dosage, including a compound described and claimed herein in combination with an injectable carrier system. As used herein, injectable and infusion dosage forms (i.e., parenteral dosage forms) include, but are not limited to, liposomal injectables or a lipid bilayer vesicle having phospholipids that encapsulate an active drug substance. Injection includes a sterile preparation intended for parenteral use.

Five distinct classes of injections exist as defined by the USP: emulsions, lipids, powders, solutions and suspensions. Emulsion injection includes an emulsion comprising a sterile, pyrogen-free preparation intended to be administered parenterally. Lipid complex and powder for solution injection are sterile preparations intended for reconstitution to form a solution for parenteral use. Powder for suspension injection is a sterile preparation intended for reconstitution to form a suspension for parenteral use. Powder lyophilized for liposomal suspension injection is a sterile freeze dried preparation intended for reconstitution for parenteral use that is formulated in a manner allowing incorporation of liposomes, such as a lipid bilayer vesicle having phospholipids used to encapsulate an active drug substance within a lipid bilayer or in an aqueous space, whereby the formulation may be formed upon reconstitution. Powder lyophilized for solution injection is a dosage form intended for the solution prepared by lyophilization (“freeze drying”), whereby the process involves removing water from products in a frozen state at extremely low pressures, and whereby subsequent addition of liquid creates a solution that conforms in all respects to the requirements for injections. Powder lyophilized for suspension injection is a liquid preparation intended for parenteral use that contains solids suspended in a suitable fluid medium, and it conforms in all respects to the requirements for Sterile Suspensions, whereby the medicinal agents intended for the suspension are prepared by lyophilization. Solution injection involves a liquid preparation containing one or more drug substances dissolved in a suitable solvent or mixture of mutually miscible solvents that is suitable for injection. Solution concentrate injection involves a sterile preparation for parenteral use that, upon addition of suitable solvents, yields a solution conforming in all respects to the requirements for injections. Suspension injection involves a liquid preparation (suitable for injection) containing solid particles dispersed throughout a liquid phase, whereby the particles are insoluble, and whereby an oil phase is dispersed throughout an aqueous phase or vice-versa. Suspension liposomal injection is a liquid preparation (suitable for injection) having an oil phase dispersed throughout an aqueous phase in such a manner that liposomes (a lipid bilayer vesicle usually containing phospholipids used to encapsulate an active drug substance either within a lipid bilayer or in an aqueous space) are formed. Suspension sonicated injection is a liquid preparation (suitable for injection) containing solid particles dispersed throughout a liquid phase, whereby the particles are insoluble. In addition, the product may be sonicated as a gas is bubbled through the suspension resulting in the formation of micro spheres by the solid particles.

The parenteral carrier system includes one or more pharmaceutically suitable excipients, such as solvents and co-solvents, solubilizing agents, wetting agents, suspending agents, thickening agents, emulsifying agents, chelating agents, buffers, pH adjusters, antioxidants, reducing agents, antimicrobial preservatives, bulking agents, protectants, tonicity adjusters, and special additives.

The compounds according to the present invention are anticipated to act as treatment agents for benign prostatic hyperplasia as can be demonstrated by standard protocols commonly known in the field. The invention encompasses methods for treating the signs and symptoms of BPH in a subject comprising administering to a subject an effective dosage of a compound according to the present invention, whereby the BPH is treated in the subject. In the treatment of BPH, suitable dosage level (i.e, an effective amount) is from about 0.001 mg/kg to about 500 mg/kg per day, and preferably about 1 mg/kg per day. The compounds may be administered on a regimen of 1 to 4 times per day, or on a continuous basis.

As appreciated by one of skill in the art, physiological disorders may present as a “chronic” condition, or an “acute” episode. The term “chronic”, as used herein, means a condition of slow progress and long continuance. As such, a chronic condition is treated when it is diagnosed and treatment continued throughout the course of the disease. Conversely, the term “acute” means an exacerbated event or attack, of short course, followed by a period of remission. Thus, the treatment of disorders contemplates both acute events and chronic conditions. In an acute event, compound is administered at the onset of symptoms and discontinued when the symptoms disappear. As described above, a chronic condition is treated throughout the course of the disease.

One of skill in the art will appreciate that particle size can affect the in vivo dissolution of a pharmaceutical agent which, in turn, can affect absorption of the agent. “Particle size” as used herein, refers to the diameter of a particle of a pharmaceutical agent as determined by conventional techniques such as laser light scattering, laser diffraction, Mie scattering, sedimentation field flow fractionation, photon correlation spectroscopy, and the like. Where pharmaceutical agents have poor solubility, small or reduced particle sizes may help dissolution and, thus, increase absorption of the agent. Amidon et al., Pharm. Research, 12; 413-420 (1995). As described in U.S. Pat. No. 7,968,587 for the SARMs of Formula I, particles can be reduced in size by methods that include milling, grinding, micronization or by other methods known in the art. Another method for controlling particle size involves preparing the pharmaceutical agent in a nanosuspension. A particular embodiment of the present invention comprises a compound of Formula (I), or a pharmaceutical composition comprising a compound of Formula (I), wherein said compound has an average particle size less than about 20 μm or a d₉₀ particle size (i.e. the maximal size of 90% of the particles) of less than about 50 μm. A more particular embodiment comprises a compound of Formula I having an average particle size less than about 10 μm or a d₉₀ particle size of less than about 30 μm. The active ingredients may have a particle size that affects the dissolution profile of a pharmaceutical agent. Particle size, as used herein, means the diameter of a particle of active pharmaceutical ingredient as determined by light scattering or other conventional techniques.

As used herein the term “effective amount” refers to the amount or dose of a compound of Formula (I) which, upon single or multiple dose administration to the patient, provides the desired effect in the patient under diagnosis or treatment. An effective amount can be readily determined by the attending diagnostician, as one skilled in the art, by considering a number of factors such as the species of mammal; its size, age, and general health; the specific disease involved; the degree or severity of the disease; the response of the individual patient; the particular compound administered; the mode of administration; the bioavailability characteristics of the preparation administered; the dose regimen selected; and the use of any concomitant medications.

### Examples

### Example 1: In Vitro Pharmacology of the Compound of Formula II

In vitro studies demonstrated that the compound of Formula II is a potent and selective modulator of the hAR with potent agonist activity in cell-based assays and no significant cross reactivity against other nuclear hormone receptors or known drug targets across various platforms. The compound of Formula II is a selective ligand for the hAR with an inhibition constant (Ki) of 1.95 nM, and a cell-based median effective concentration (EC50) of 1.25 nM, with demonstrated agonist activity. The binding affinity for hAR compared to other nuclear hormone receptors was >500-fold (see Table 3).

TABLE 3

| Assay | TT701 | |
|---|---|---|
| AR Ki (nM) | 1.95 | |
| AR C2C12 | 1.25 | |
| EC50 (nM) | | |
| ER, GR, PR | >3,000 | |
| MR(IC50/Ki)(nM) | | |
| Muscle LA ED | 3 | mpk |
| mg/kg | | |
| Bone Eff (BM) | 2 | mpk |
| Rat uterine risk | 20 | mpk |
| Rat SV/Prost risk mgs/kg | >30 | mpk |

| | |
|---|---|
| Rat F % | 63 |
| Dog F % | 58 |
| MOS (AUC) | 28X_{dog} + 71X_{cat} |

### Example 2: Structural, Chemical, and Pharmacological Characteristics of the Compound of Formula II

Structural Characteristics of the Compound of Formula II:

The compound of Formula II belongs to a nonsteroidal THCI scaffold that is structurally distinct from the cholesterol-derived steroidal scaffolds. The compound of Formula II has weak affinity to serum hormone binding globulin (none detected at 10 μM) and is not metabolized by 17-beta-Hydroxysteroid Dehydrogenase Type 2 class of enzymes. The x-ray crystallography structure of the compound of Formula II-bound AR illustrates some key differences in the contact sites within the active pocket relative to that of dihydrotestosterone-bound AR.

### Example 3: In Vitro Activity of the Compound of Formula II on LnCAP Cells

The compound of Formula II has weak agonist activity in in vitro prostate LnCAP cells (androgen-sensitive human prostate adenocarcinoma cells) being at least 46 fold weaker than the synthetic testosterone R188. Comparisons of the compound of Formula II with the synthetic Testosterone R1881, showed that in vitro using human prostate cancer cells the LY compound is less androgenic than R1881. In contrast the biochemical binding affinity to the human Androgen receptor (Ki in nM) is only modestly reduced. The ability of the compound of Formula II to bind to the Androgen receptor and yet have a very weak agonist activity in gene expression compared to the synthetic Testosterone R188, suggests that the presence of the compound of Formula II may interfere or reduce AR activity of endogenous Testosterone (see Table 4).

TABLE 4

| | | LnCAP Gene Expression EC50 (nM) | | |
|---|---|---|---|---|
| | AR Ki (nM) | PSA | AR | CLUSTERIN |
| R1881 | 0.38 | 0.034 | 0.035 | 0.37 |
| Compound | 1.95 | 2.64 | 1.64 | >100 |
| of Formula | | | | |
| II | | | | |
| Fold Diff | ~5X | ~77X | ~46X | ~270X |

### Example 4: Anabolic and Androgenic Effects of the Compound of Formula II in Different Animal Studies

The effect of short and long term treatment of the compound of Formula II on prostate size of rats and dogs was studied. As part of the toxicology program, rats and dogs were treated with escalating doses of the compound of Formula II for 1, 3, 6 or 12 months depending on the study or species and examined for prostate size and histologically for prostate atrophy. The data is shown in Tables 5 and 6 below.

TABLE 5
Rat Data

| | Treatment duration (months) | | | | | | | | |
|---|---|---|---|---|---|---|---|---|---|
| | | | | | | | Male Fertility | | |
| | 1+ | | | 6+ | | | (3 months) | | |
| | Dose (mg/kg/day) | | | | | | | | |
| | 15 | 150 | 1500 | 15 | 150 | 1500 | 3 | 30 | 1000 |
| Prostate | “” | “” | “” | “” | ^{↓}30% | ^{↓}34% | “” | ^{↓}23% | ^{↓}39% |
| weight(mean % | | | | | | | | | |
| decrease) | | | | | | | | | |
| Prostate | “” | “” | “” | “” | | 4/15 | “” | “” | “” |
| atrophy (no | | | | | | | | | |
| affect/no | | | | | | | | | |
| examined) | | | | | | | | | |
| Male group | 35733 | 72283 | 102337 | 15902 | 34132 | 82690 | 10954 | 49734 | 56009 |
| mean AUC₀₋₂₄ | | | | | | | | | |

TABLE 6
Dog Data

| | Treatment duration (months) | | | | | | | | |
|---|---|---|---|---|---|---|---|---|---|
| | 1+ | | | 6+ | | | 12 | | |
| | Dose (mg/kg/day) | | | | | | | | |
| | 3 | 30 | 150 | 3 | 30 | 150 | 3 | 10 | 100 |
| Prostate | “” | “” | “” | ^{↓}63% | ^{↓}66% | ^{↓}75% | ^{↓}60 | ^{↓}62% | ^{↓}80% |
| weight(mean % | | | | | | | | | |
| decrease) | | | | | | | | | |
| Prostate | “” | “” | 1/4 | 4/4 | 4/4 | 4/4 | 2/4 | 3/4 | 4/4 |
| atrophy (no | | | | | | | | | |
| affect/no | | | | | | | | | |
| examined) | | | | | | | | | |
| Male group | 17984 | 61674 | 46528 | 6492 | 44448 | 53032 | 3641 | 13408 | 22582 |
| mean AUC₀₋₂₄ | | | | | | | | | |

“” no effect observed.
Vehicle 80% PEG 1350, 20% vitamin E TPGS (w/v)

These data demonstrate that treatment with the compound of Formula II at different doses decreased prostate weight in both rats and dogs. The loss in weight was more pronounced in dogs relative to rats, and in dogs decreases of 60-75% were observed by 3 months. In addition, histological examination showed prostate atrophy in 50 to 100% of the animals treated for 6-12 months.

Importantly, there is a lack of androgenic effect on the prostate with very high doses of the compound of Formula II in both rats and dogs. The reductions in the weight and/or atrophy of the prostate are consistent with the antagonistic properties of the compound of Formula II on androgenic effects on the prostate.

### Example 5: Anabolic and Androgenic Effects of the Compound of Formula II in the Rat Osteopenic Orchidectomized (ORX) Model

An orchidectomized rat model was used to examine the anabolic and androgenic effects of the compound of Formula II in the absence of endogenous testosterone in animals. Orchidectomized (ORX) and sham-operated Wistar male rats were used (orchidectomized at 8 weeks of age and allowed to waste for 4 weeks). The rats were maintained on a 12 hr light/dark cycle at 22° C. with ad lib access to food (TD 5001 with 0.95% Ca and 0.67% P, Teklad, Madison, Wis.) and water. Rats were randomized and placed into treatment groups (n=6) based on body weight. Route of administration for all groups except testosterone enanthate (TE was given sub-cutaneously) was oral. At the end of 2 weeks of daily rats were euthanized, weighed & tissue harvested. Levator ani, prostates, and seminal vesicles were collected from each animal. Results are plotted as means±SE.

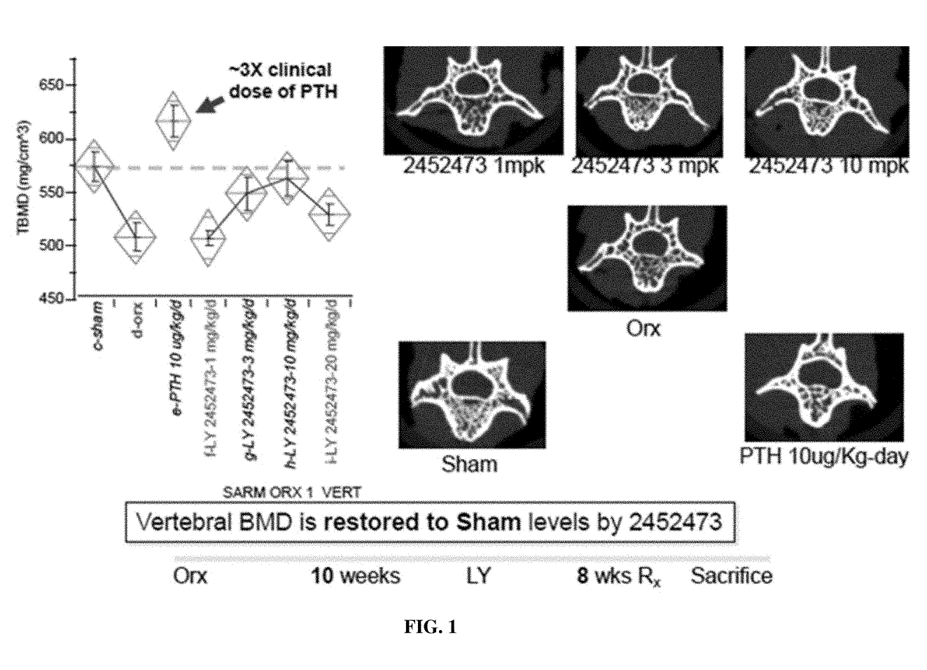

The results are illustrated in FIG. 1, daily oral administration of the compound of Formula II led to a dose-dependent increase in vertebral bone mineral content (BMC), cross-sectional area, and bone mineral density (BMD). A significant increase in the bone anabolic biomarkers, rat procollagen type 1 amino-terminal propeptide (P1NP), and periosteal alkaline phosphatase, were observed after 2 and 8 weeks of treatment.

### Example 6: Anabolic Effects of the Compound of Formula II on the Levator Ani- and Bulbocavernous Muscle in the ORX Model

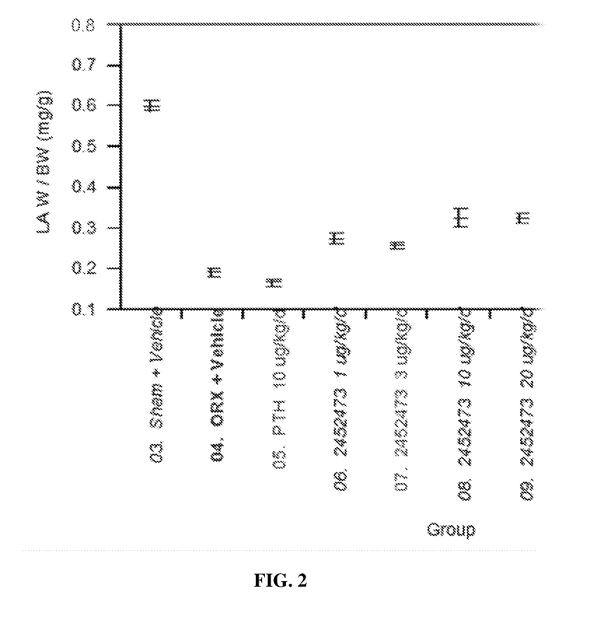

The anabolic effects of the compound of Formula II on the Levator ani- and bulbocavernous muscle in the delayed ORX model were examined. Following the androgen deficiency in the ORX model, the Levator ani- and bulbocavernous muscle weights decreased in size. Following treatment with the compound of Formula II for 8 weeks, the Levator ani- and bulbocavernous muscle weights increased significantly with doses of 1 to 3 mg/kg/day doses. These data indicate that the compound of Formula II has positive anabolic effects on the Levator ani and bulbocavernous muscle in testosterone deficient animals. The results are graphically represented in FIG. 2 and tabulated in Table 7.

TABLE 7

| Subject | Dose (ug/kg/d) | Number | Mean | Std Error Mean |
|---|---|---|---|---|
| 3 | Sham + Vehicle | 7 | 0.599700 | 0.01204 |
| 4 | ORX + Vehicle | 7 | 0.192057 | 0.01180 |
| 5 | PTH 10 | 6 | 0.166467 | 0.00913 |
| 6 | compound of | 6 | 0.274917 | 0.01522 |
| | Formula II - 1 | | | |
| 7 | compound of | 7 | 0.257529 | 0.00773 |
| | Formula II - 3 | | | |
| 8 | compound of | 7 | 0.323743 | 0.02138 |
| | Formula II - 10 | | | |
| 9 | compound of | 7 | 0.325043 | 0.01218 |
| | Formula II - 20 | | | |

The compound of Formula II demonstrated a robust indicator of muscle and bone loss associated with androgen deprivation. In 2 and 8 week studies, the compound of Formula II demonstrated the ability to increase intrapelvic skeletal muscle wet weight, restore bone loss, and improve bone strength in the cortical site and femoral neck.

### Example 7: The Androgenic Effects of the Compound of Formula II in the ORX Rat Model

FIGS. 1 and 2 illustrate the effect of the compound of Formula II treatment for 2 or 8 weeks on the prostate weight and seminal vesicles in the delayed ORX rat model. The weights and seminal vesicals of orchidectomized rats decreased significantly. Treatment of delayed ORX rats with increasing doses of the compound of Formula II had no effect on the prostate or seminal vesical weights. These data clearly demonstrate that doses of up to 20-30 mg/kg do not have androgenic activity on the prostate.

Anabolic activity on muscle and bone induced by the compound of Formula II treatment was observed in the absence of androgenic related effects on prostate weight and histology or on seminal vesicles weight that confirmed the ‘prostate sparing’ effects of the compound of Formula II.

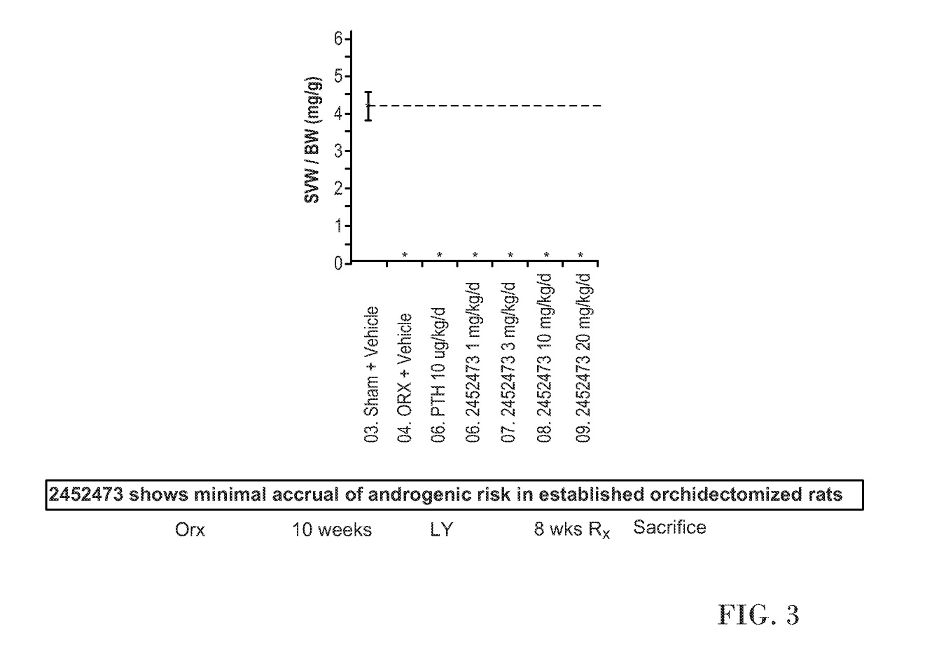

The data in FIG. 3 shows that TT701 treatment in the ORX rat shows minimal accrual SV/Prostate risk. Thus, anabolic activity on muscle and bone induced by TT701 treatment was observed in the absence of androgenic related effects on prostate weight and histology or on seminal vesicles weight, which confirms the prostate sparing effects of TT701.

### Example 8: Effect of the Compound of Formula II on Testosterone Induced Prostate Growth in Delayed ORX Rat Model

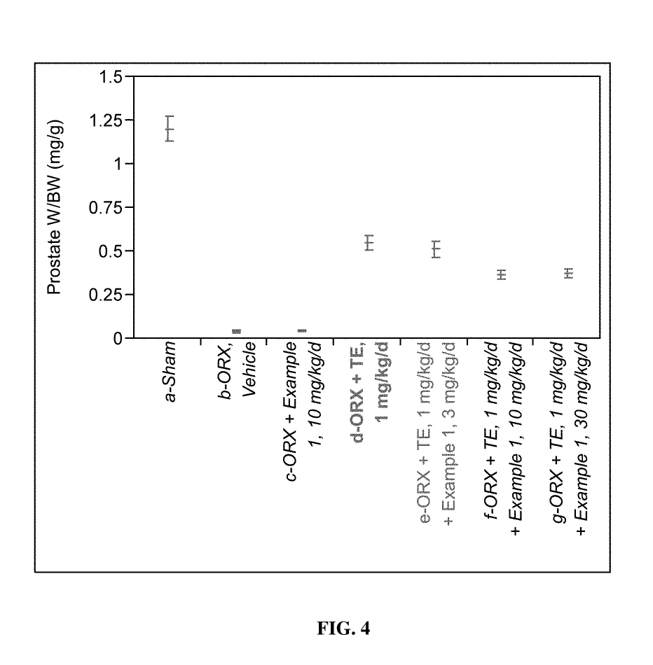

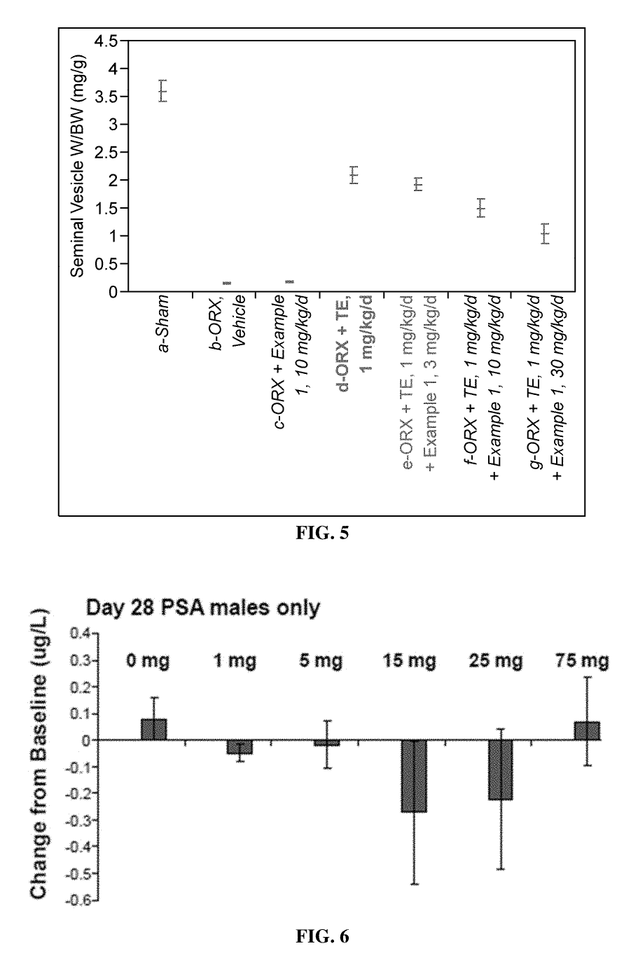

FIGS. 4 and 5 illustrate the effect of orchidectomized rats treated with Testosterone (1 mg/k/d) alone for 2 weeks showed an increase in prostate weight and seminal vesical weight, respectively. The combination with Testosterone Enanthate (TE) (1 mg/kg/d) and the compound of Formula II at 3, 10 and 30 mg/kg/d demonstrated a decreasing prostate weight and seminal vesical weights normalized to body weight, compared to the prostate and seminal vesical weights induced by TE alone after 2 weeks of co-treatment. These data demonstrated that the compound of Formula II antagonized the androgenic effects of testosterone on the prostate. Table 8 contains the data on the effect on prostate weight of the treatment with TE and the compound of Formula II. Table 9 contains the date on the effect on seminal vesical weight of the treatment with T and the compound of Formula II.

TABLE 8
Means Comparisons of prostate weights (Comparisons with a control
using Dunnett's Method Control Group = d-ORX + TE, 1 mg/kg/d
|d| 2.69715 Alpha 0.05

| Group | | Abs(Dif)- | |
|---|---|---|---|
| No | Group | LSD | p-Value |
| 1 | Sham | 0.509 | <.0001* |
| 2 | ORX + TE, 1 mg/kg/d | −0.15 | 1.0000 |
| 3 | ORX + TE, 1 mg/kg/d + Example 1, | −0.11 | 0.9774 |
| | 3 mg/kg/d | | |
| 4 | ORX + TE, 1 mg/kg/d + Example 1, | 0.025 | 0.0167* |
| | 30 mg/kg/d | | |
| 5 | ORX + TE, 1 mg/kg/d + Example 1, | 0.036 | 0.0099* |
| | 10 mg/kg/d | | |
| 6 | ORX, Vehicle | 0.356 | <.0001* |
| 7 | ORX + Example 1, 10 mg/kg/d | 0.357 | <.0001* |

Positive values show pairs of means that are significantly different than TE alone group

TABLE 9
Means Comparisons of seminal vesicle wet weights
(Comparisons with a control using Dunnett's Method):
Control Group = d-ORX + TE, 1 mg/kg/d
|d| 2.69715 Alpha 0.05

| Group | | Abs(Dif)- | |
|---|---|---|---|
| No | Group | LSD | p-Value |
| 1 | Sham | 0.979 | <.0001 |
| 2 | ORX + TE, 1 mg/kg/d | −0.52 | 1.0000 |
| 3 | ORX + TE, 1 mg/kg/d + Example 1, | −0.34 | 0.8628 |
| | 3 mg/kg/d | | |
| 4 | ORX + TE, 1 mg/kg/d + Example 1, | 0.078 | 0.0187 |
| | 10 mg/kg/d | | |
| 5 | ORX + TE, 1 mg/kg/d + Example 1, | 0.536 | <.0001 |
| | 30 mg/kg/d | | |
| 6 | ORX + Example 1, 10 mg/kg/d | 1.411 | <.0001 |
| 7 | ORX, Vehicle | 1.422 | <.0001 |

Positive values show pairs of means that are significantly different.

### Example 9: Clinical Studies with the Compound of Formula II

Six clinical studies were completed with the compound of Formula II (Table 10): five Phase 1 clinical trials (Studies GPBA, GPBC, GPBG, GPBF, and GPEA) and one Phase 2 clinical trial (Study GPEC). A total of 353 subjects have been exposed to the compound of Formula II in these completed clinical studies. In Studies GPEA and GPEC, the compound of Formula II was orally co-administered with tadalafil.

TABLE 10
Completed Clinical Studies with the compound of Formula II

| Study | |
|---|---|
| Number | |
| (Section) | Study Design (N) |
| GPBA | Phase 1, randomized, placebo-controlled, double-blind, single- |
| | dose (5, 25,125, 250, 500 or 1000 mg/day), incomplete-cross- |
| | over, dose-escalation study in healthy men and postmenopaus- |
| | al women (N = 29) |
| GPBC | Phase 1, randomized, placebo-controlled, double -blind, multi- |
| | ple-dose (1, 5,15, 25, 75 mg/day), dose-escalation study of |
| | the compound of Formula II inhealthy subjects for 28 days |
| | (N = 44) |
| GPBG | Single-center, open-label, 2-period, fixed-sequence study to |
| | assess the effects of multiple oral doses of the compound of |
| | Formula II (5 mg/day) on simvastatin and simvastatin acid |
| | activity in healthy subjects (N = 16) |
| GPBF | Phase 1, open-label, 4-period crossover bioavailability study |
| | conducted in healthy male subjects to evaluate the pharmaco- |
| | kinetics of the compound of Formula II and tadalafil (N = 24) |
| GPEA | Phase 1, randomized, open-label, 4-period crossover bioavail- |
| | ability study conducted in healthy male subjects to evaluate |
| | the pharmacokinetics of the compound of Formula II and |
| | tadalafil (N = 24) |
| GPEC | Phase 2, randomized, double-blind, parallel, controlled clini- |
| | cal trial in patients with ED who had an incomplete response |
| | to tadalafil; 5 dosing arms: the compound of Formula II 1 |
| | mg or 5 mg + tadalafil 5 mg, the compound of Formula II 5 |
| | mg, tadalafil 5 mg, tadalafil 10 mg, (N = 234) |

### Example 10: Safety Assessment of TT701

Study GPBA detected a QT signal using a concentration-response analysis which showed a statistically significant positive relationship between TT701 and QTcF prolongation. At TT701 concentrations found with doses of 250 mg or greater, the mean QTcF prolongation was greater than 10 msec. The risk for clinically significant QT prolongation at doses <250 mg can be excluded. Review of ECG data in study GPEC did not show evidence of clinically meaningful changes associated with TT701.

In study GPEC, treatment with OPK-88004 was associated with decreased HDL cholesterol levels and apolipoprotein A1, whereas observed decreases in total cholesterol, triglycerides and LDL cholesterol levels were not considered clinically meaningful.

In clinical studies with TT701, no significant increases in total bilirubin, GGT, or alkaline phosphatase were observed at any dose. Transient elevation of liver transaminases (ALT or AST) observed in 14 subjects in phase 1 studies were not considered to be clinically significant by the investigator, and none were captured as AEs. In a phase 2 study, three subjects had transient abnormal AST or ALT, with levels in two subjects receiving OPK-88004 being ≥2×ULN and in one subject being ≥3×ULN. None of the elevations were considered to be clinically significant by the investigator and none were captured as AEs. All of the subjects with elevated liver transaminase levels completed the respective study.

In Study GPEC, endocrine-related parameters evaluation measured in the 12-week study in elderly men included total and free testosterone, SHBG, estradiol, follicle-stimulating hormone (FSH), and LH and semen analysis. A clinically meaningful decrease was observed for testosterone, accompanied by a reduction of SHBG,

TABLE 11
OPK-88004 Noncompartmental Pharmacokinetic Paramenters following
a Single Oral Dose of OPK-88004 to Healthy Subjects (Study GPBA)
Geometric Mean (CV %)

| | 5 mg | 25 mg | 125 mg | 250 mg | 500 mg | 1000 mg |
|---|---|---|---|---|---|---|
| N | 8 | 7 | 7 | 7 | 7 | 6 |
| Cₘₐₓ(ng/mL) | 25.9 | 123 | 428 | 589 | 817 | 910 |
| | (53) | (34) | (36) | (70) | (26) | (39) |
| Tₘₐₓ^{a}(h) | 5.00 | 4.00 | 5.00 | 5.00 | 4.00 | 4.50 |
| | (3.00-6.00) | (3.00-5.00) | (4.00-5.00) | (3.00-10.02) | (3.00-5.00) | (2.00-10.02) |
| T_{1/2}^{b}(h) | 27.1 | 26.6 | 39.4^{c} | 31^{c} | 55.1^{d} | 41.7 |
| | (18.1-53) | (17.5-35.0) | (14.4-114) | (17.3-65.0) | (22.7-138) | (21.1-79.9) |
| AUC₍₀₋₎ | 458 | 2110 | 8590^{c} | 17500^{c} | 29400^{d} | 24100 |
| | (36) | (20) | (111) | (29) | (68) | (86) |

Abbreviations: Cₘₐₓ = maximum observed drug concentration; tₘₐₓ = time of maxium observed drug concentration; AUC (0-) = area under the concentration-time curve from 0 to infinity;
^{a}Median (range);
^{b}Geometric mean (range);
^{c}n = 6;
^{d}n = 5.

### Example 11: The Effect of the Compound of Formula II on Prostate Antigen

In Study GPBC, the PSA test indicated a neutral to negative effect (decreasing PSA) on the prostate in response to increased dosing in the dose escalation study in healthy subjects (1 mg to 75 mg daily dose for 28 days) of the compound of Formula II. The results of this study are illustrated in FIG. 6.

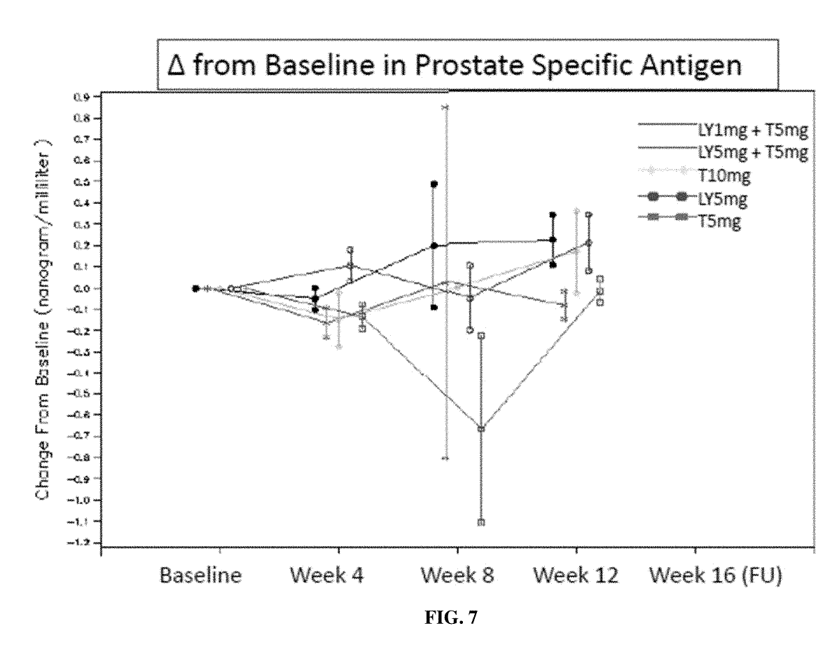

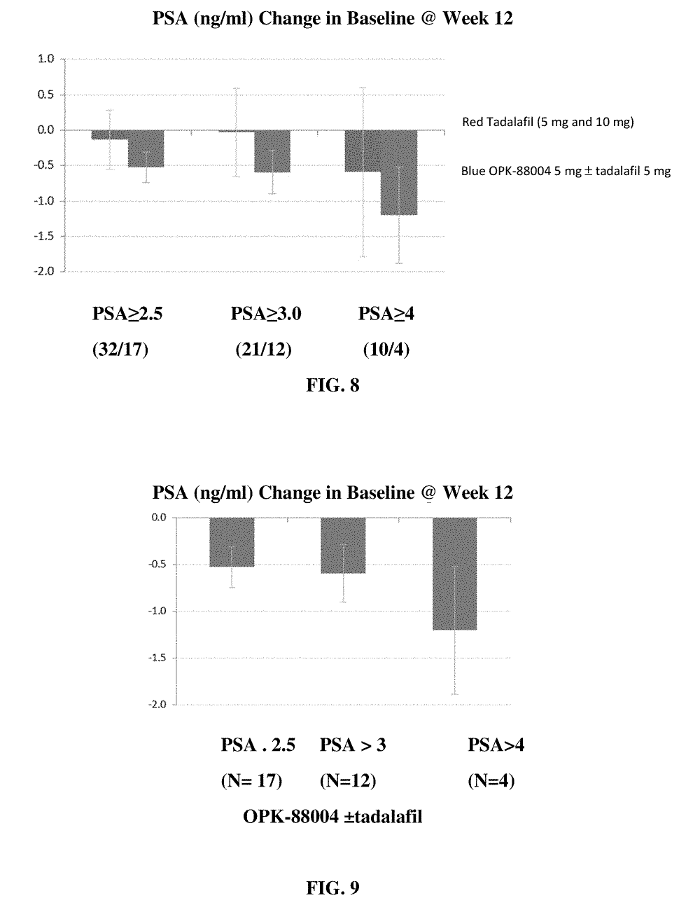

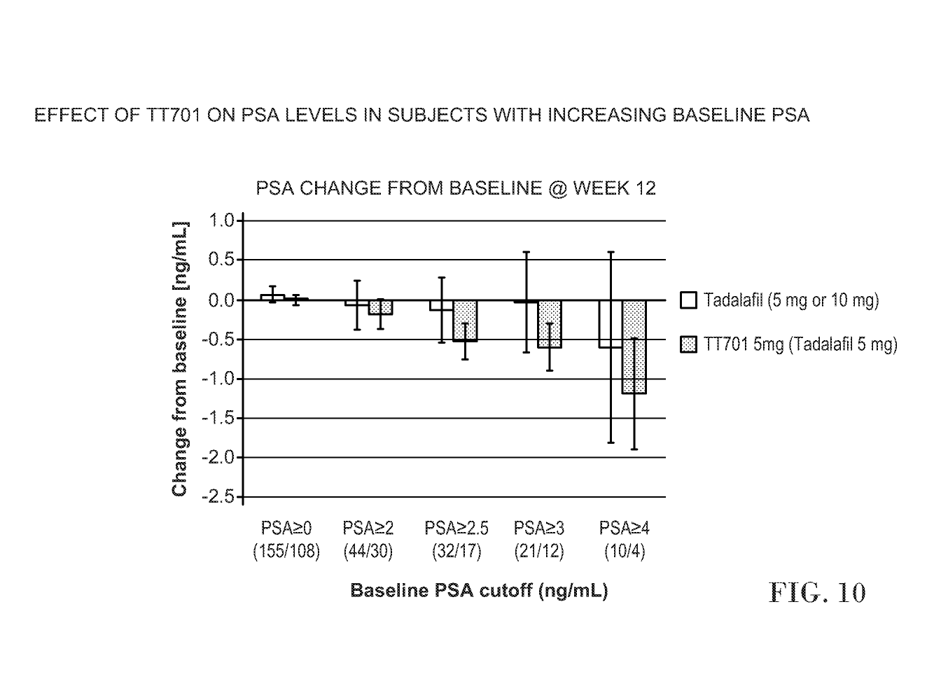

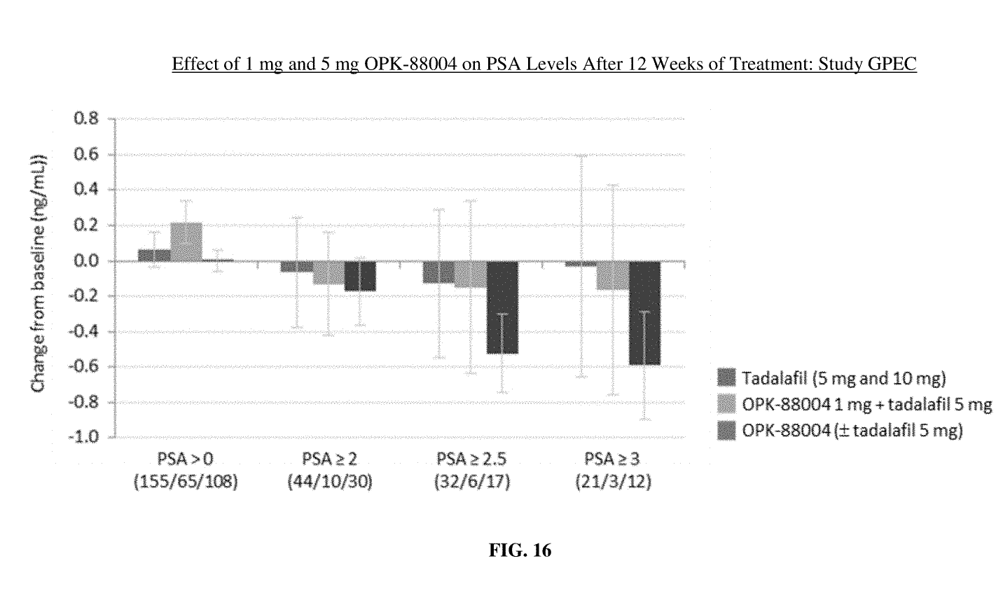

In Study GPEC, treatment with the compound of Formula II at doses of 1 mg and 5 mg for 12 weeks was not associated with increases in PSA. The results of this study are illustrated in FIG. 7. Also in this study and significantly for the treatment of BPH, in this study in healthy aging males who had symptoms of erectile dysfunction and were failing on tadalafil treatment, TT701 (OPK-88004, 5 mgs) alone or in combination with 5 mgs or 10 mgs of tadalafil and in patients having PSA levels of greater than about 2.5 ng/mL, unexpectedly showed a significant decrease in PSA levels relative to baseline. (see FIG. 8 and FIG. 9). FIG. 10 shows the changes in PSA levels in patients having symptoms of erectile dysfunction and who were failing tadalafil treatment being administered OPK-88004 (TT701) alone or combination with tadalafil (5 mgs alone or 5 mg/5 mg combination) where the PSA horizontal scale is broadened. This figure shows you begin to see a decrease in PSA levels at about 2.0 PSA upon treatment with OPK-88004. FIG. 16 shows the changes in PSA levels in patients being administered tadalafil alone (5 mg and 10 mg) or OPK-88004 (TT701) (1 mg or 5 mg) in combination with tadalafil (5 mg) after 12 weeks of treatment.

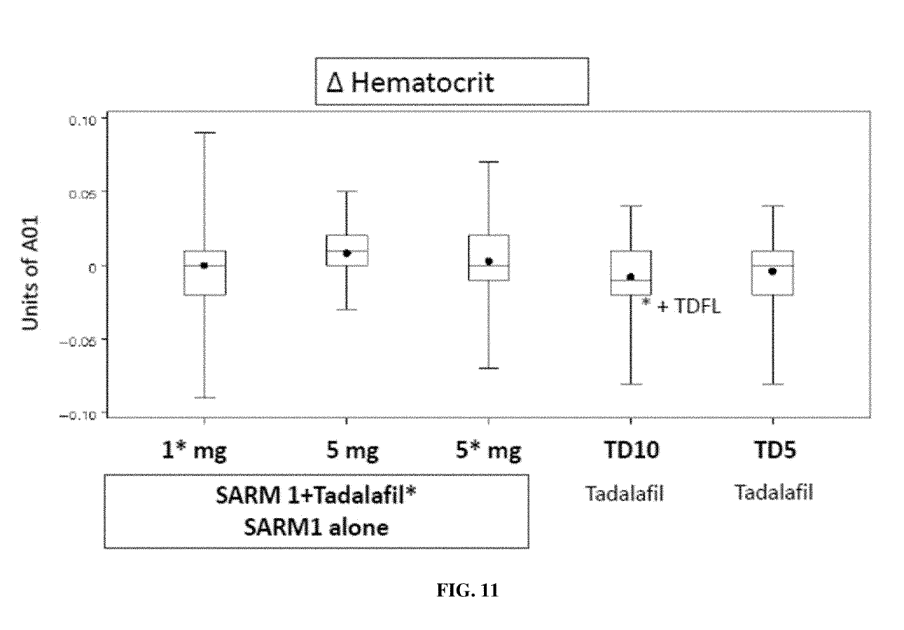

To date, no immunotoxicity safety signal has been observed. It is believed that because the compound of Formula II is a small molecule, it is not expected to be immunogenic, and an immunogenicity assay has not been developed. Androgen-induced erythrocytosis and resulting polycythemia is thought to be a significant limitation to androgen therapy, and has been shown to manifest in the first 3 months of treatment. A statistical analysis of hemoglobin and hematocrit showed no increases in these parameters at any of the compound of Formula II doses tested. FIG. 11 shows that TT701 causes no change in Hematocrit with the doses tested. Examination of vital sign data from clinical studies of TT701 did not yield any significant changes in systolic blood pressure, diastolic blood pressure, or heart rate. Subjects in Study GPBC received visual acuity, visual field assessment, and fundoscopy examinations as well as an Ocular Surface Disease Index test before and after receiving TT701. There were no clinically relevant changes in these assessments.

### Example 12: Anabolic Effects of the Compound of Formula II

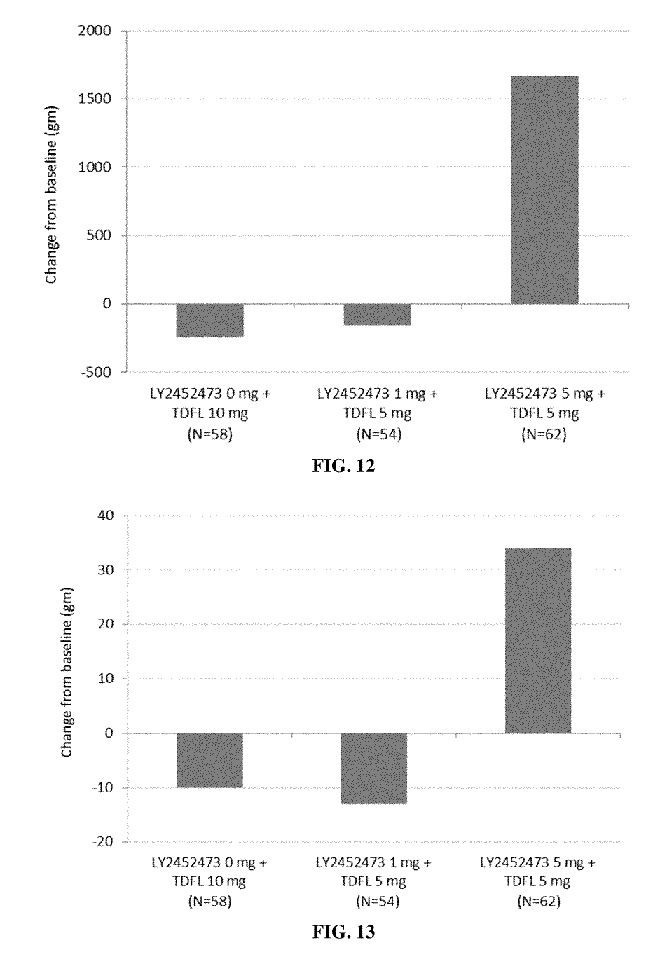

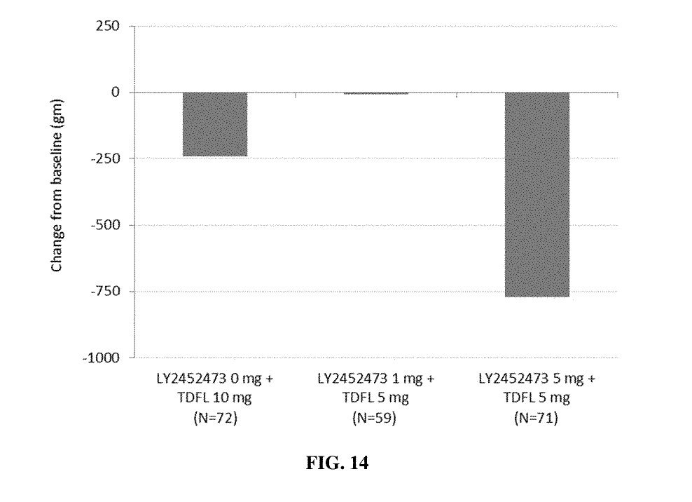

The available data indicate that the compound of Formula II may have had the agonist effects of an androgen via decreased fat mass and increased LBM. In a multiple dose study (Study GPBC), healthy subjects exposed to the compound of Formula II for 28 days demonstrated clinically and statistically significant increases in LBM and calf area (by CT). The Phase 2 Study for ED also included exploratory measures for lower extremity muscle strength and power, LBM and fat mass. In this study, 12 weeks of daily treatment with the compound of Formula II indicated that the compound of Formula II may have had the agonist effects of an androgen via decreased fat mass and increased LBM. Patients receiving a combination treatment of 5 mg the compound of Formula II+5 mg tadalafil had a reduction of fat body mass and an increase (improvement) of LBM compared with patients receiving 10 mg tadalafil. Lower extremity muscle power, as measured by the stair climb, was increased (improved) in patients receiving a combination treatment of 5 mg the compound of Formula II+5 mg tadalafil compared with patients receiving 10 mg tadalafil. The results are illustrated in FIGS. 12, 13, and 14.

FIG. 11 illustrates the fat Mass mean change from baseline to week 12: Study GPEC.

### Example 13: Clinical Study for BPH in Patients with and without PSA Levels ≥2.5 ng/mL

Patients will also receive, in separate arms, doses of SARM (OPK-88004, 5 mgs 1×/day) and tadalafil (5 mg 1×/day) versus placebo. The patients can achieve a 1-5%, 6-10%, 11-20%, 21-40%, 41-60% or greater reduction in prostate volume relative to normal prostate size/volume. The other clinical measurements for the treatment of the signs and symptoms of BPH include those described herein such as measuring, as a primary endpoint, the International Prostate Symptom Score (IPSS). The IPSS is a four week recall questionnaire administered to both a placebo group and after randomization. The IPSS is used to assess the severity of irritative symptoms such as frequency, urgency or straining as well as obstructive symptoms such as incomplete emptying, stopping and starting, weak stream and straining or pushing. The IPSS' numeric scores can range from 0 to 35 where higher scores are indicative of a more severe condition. A secondary endpoint in clinical studies conducted for BPH includes measuring the maximum urinary flow rate (Qmax). In addition, PSA will be measured in all patients being treated in all arms of the study(ies).

### Example 14: Clinical Studies in Patients with BPH and Having Sexual Symptoms, Fatigue/Low Vitality and Physical Dysfunction

Patients will also include those (1) serum testosterone, measured by LC-MS/MS, <300 mg/dL and/or free testosterone by equilibrium dialysis <60 pg/mL; (2) self-reported sexual dysfunction (IIEF score <25 or sexual desire score <7), (3) fatigue (FACIT-F score >30), or physical dysfunction (self-reported difficulty in walking a ¼ mile or climbing two flights of stairs, short physical performance battery score 4 to 9); and (4) ability to understand and the willingness to sign a written informed consent document.

The clinical study will evaluate the efficacy and safety of TT701 in improving the symptoms of androgen deficiency (sexual symptoms, fatigue/low vitality and physical dysfunction) in men with BPH. The primary outcome measure is based upon answers to the Harbor-UCLA 7-day sexual function questionnaire and secondary outcome measures include answers to disease specific quality of life; functional assessment of fatigue; lean body mass; muscle strength and a continuous scale physical function assessment. The subjects are randomized to receive placebo or oral SARM (TT701) 10 mgs 2× per day (Dose 1) or 5 mgs 2× per day (Dose 2). TT701 is a selective androgen receptor modulator which is agonist on the muscle and which spares the prostate.

It is believed that because of the reduction of prostate volume by the SARM of the claimed invention, alone or in combination with a PDE-5 inhibitor, it will have a longer duration of efficacy relative to other symptomatic drugs such as alpha blockers or PDE-1 inhibitors.

### Example 15: Clinical Studies Administering TT701 (OPK88004) in Patients with Lower Urinary Tract Symptoms (LUTS) Secondary to BPH

Study Design

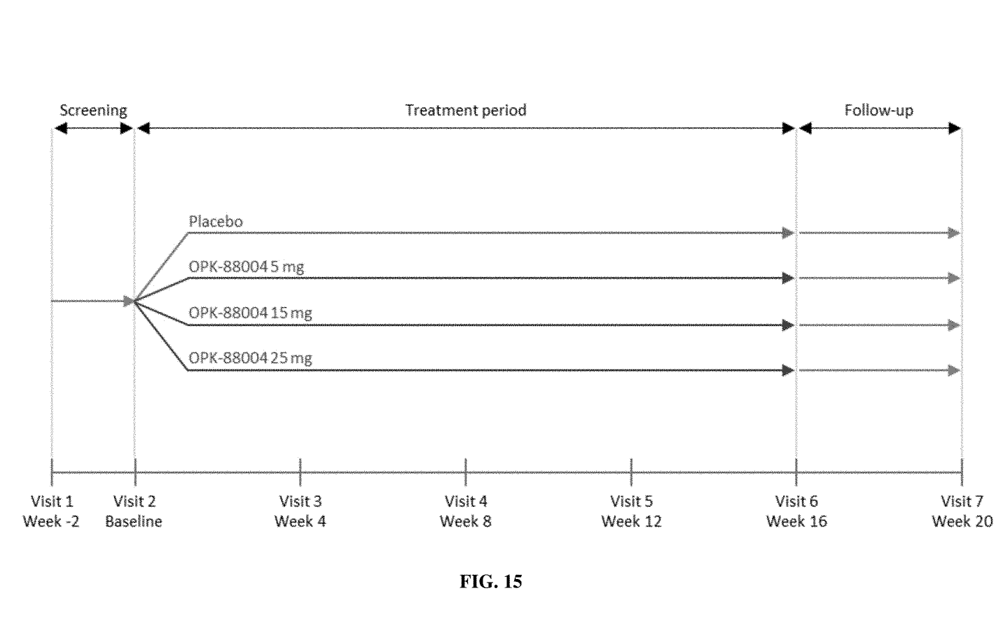

The clinical study will evaluate the treatment of approximately 100 male subjects with lower urinary tract symptoms (LUTS) secondary to BPH by administering TT701 in 5 mg, 15 mg or 25 mg, or matching placebo daily for 16 weeks (FIG. 15). Subjects will be stratified 1:1 according to baseline prostate volume greater or less than 60 cm3 and balanced for IPSS score, and will be randomized 1:1:1:1.

The patient population will consist of subjects with moderate to severe BPH-LUTS, including prostate volume (determined by TRUS) >40 cm³ and <80 cm³ international prostate symptom score (questions 1-7, IPSS) ≥13 and IPSS bother score (IPSS QoL question 8>3) and bladder outlet obstruction as defined by a urinary peak flow rate (Qₘₐₓ) between 4 and 15 mL/s on a voided volume (V_{comp}) of at least 125 mL. Subjects will be 45 to 75 years of age and have serum PSA >1.5 and <10.0 ng/mL at screening. Subjects with potential alternative causes of symptoms or prostatic enlargement will be excluded.

The primary efficacy endpoints will be prostate volume and serum PSA following 16 weeks treatment. Size will be the most sensitive marker for an effect on the prostate, and reduction in prostate volume may underlie improvement in urinary flow and associated symptoms. The reduction in prostate size may be measurable as early as three months after starting treatment, and the effect will increase further and is thereafter maintained with continued treatment.

The exposure within the proposed clinical dose range will result in clinically meaningful reductions. Reduction in prostate volume of approximately 20% will be reported along with clinically significant improvement in clinical scores and maximum urinary flow rate. In addition, reduction of PSA levels will be shown.

Secondary efficacy endpoints will include post-void residual volume (PVR) and uroflowmetry parameters including peak flow rate (Qmax), mean flow rate (Qave) and PVR determined by ultrasound. Symptoms, as assessed by IPSS scores, will also be investigated at end-of-treatment as an exploratory endpoint. Longer treatment than 16 weeks may be required to detect an effect on these subjective assessments. Androgenic effects of OPK-88004 will be assessed by measurements of lean body and fat mass (by DEXA).

Safety Assessment

Routine safety measurements will be conducted during this trial including monitoring of the following: adverse events; clinical laboratory assessments collected at weeks 4, 8 and 16; vital signs, weight and physical exam; ECGs at weeks 8 and 16.

### Example 16: Clinical Studies Administering TT701 (OPK88004) and Tadalafil in Patients with Moderate-to-Severe BPH-LUTS and Enlarged Prostates

The clinical study will evaluate the treatment of approximately 100 male subjects moderate-to-severe BPH-LUTS and Enlarged Prostates by administering OPK88004 in 5 mg, 15 mg, or 25 mg and tadalfil in 5 mg, 15 mg, or 25 mg in a fixed-dose combination oral dosage form daily for up to 6 months or more.

The primary efficacy endpoint in this clinical study will be changes in the validated symptom index International Prostate Symptom Score (IPSS) after 2 years treatment. Changes in peak urinary flow rate (Qₘₐₓ) will be a secondary efficacy endpoint.

OPK88004 will reduce prostate size and prevent progression of LUTS secondary to BPH and reduce the risk of urinary retention. Tadalafil will lower urinary tract smooth muscle relaxation via PDE5 inhibition within 4 weeks. These two agents with separate mechanisms of action will result in at least additive or more than additive clinical improvements in the symptoms of BPH. The combination therapy will to provide more timely relief of symptoms in men with BPH-LUTS and enlarged prostate due to the rapid onset of action with tadalafil, and subsequent additive effects as prostate size is reduced by OPK-88004.

The combination therapy of OPK-88004 and tadalafil will show surprising unexpected properties compared to either drug alone. The present invention is inclusive of improving any one of the signs or symptoms of BPH using a compound of formula I or II or a combination of a compound of formula I or II with a PDE5 inhibitor in a patient in need of treatment thereof. The present invention also relates to a fixed dosage combination of a compound of formula I or II with a PDE5 inhibitor. Such a fixed dose dosage form may be in the form of a capsule or tablet having the amount of active ingredients for a compound of formula I or II and for a PDE5 inhibitor as described in the specification. The combination of a compound of formula II with tadalafil for the treatment of BPH is a significant improvement over the treatment of BPH using tadalafil alone in BPH patients and may additionally result in additional positive anabolic effects including the maintenance of muscle mass and muscle strength.

1. A method of treating the signs and symptoms of benign prostatic hyperplasia (BPH) in a subject comprising administering a therapeutically effect amount of at least one tetrahydrocyclopenta[b]indole compound to a subject in need thereof, wherein the tetrahydrocyclopenta[b]indole compound has Formula I: 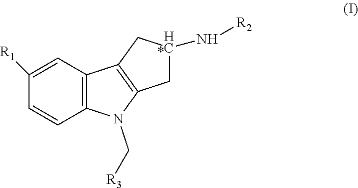
wherein the C* atom may be R, S or R/S configuration;
R₁ represents cyano, —CH═NOCH₃, —OCHF₂, or —OCF₃;
R₂ represents —COR₂ₐ or —SO2R_{2b};
R₂ₐ represents (C₁-C₄)alkyl, (C₁-C₄)alkoxy, cyclopropyl, or —NRaRb;
R_{2b} represents (C₁-C₄)alkyl, cyclopropyl, or —NraRb;
Ra and Rb each independently is H or (C₁-C₄)alkyl; and
R₃ represents a heteroaryl group selected from the group consisting of pyridinyl, pyrimidinyl, pyrazinyl, pyridazinyl, thiazoloy isothiazolyl, and thiadiazolyl, each optionally substituted with 1 or 2 substituents independently selected from the group consisting of methyl, ethyl, bromo, chloro, fluoro, —CHF₂, —CF₃, hydroxyl, amino and —NHCH₂CO₂H, or a pharmaceutically acceptable salt thereof.

2. The method according to claim 1, wherein:
(a) R₁ is CN, —CH═NOCH₃ or —OCF₃;
(b) R₂ is —COR₂ₐ or —SO2R_{2b} wherein R₂ₐ is (C₁-C₄)alkyl, (C₁-C₄)alkoxy, cyclopropyl, or —N(CH₃)₂ and R_{2b} is (C₁-C₄)alkyl, cyclopropyl, —N(CH₃)₂ or —N(C₂H₅)₂;
(c) R₂ is —COR₂ₐ or —SO₂R_{2b} and R₂ₐ is ethyl, isopropyl, methoxy, ethoxy, propoxy, isopropoxy, isobutoxy, tert-butoxy, cyclopropyl, or —N(CH₃)₂ and R_{2b} is methyl, ethyl, propyl, cyclopropyl, —N(CH₃)₂ or —N(C₂H₅)₂;
(d) R₂ is —COR₂ₐ and R₂ₐ is selected from ethyl, isopropyl, methoxy, ethoxy, propoxy, isopropoxy, isobutoxy, tert-butoxy, cyclopropyl, or —N(CH₃)₂;
(e) R₂ is —SO₂R_{2b}, and R_{2b} is methyl, ethyl, propyl, cyclopropyl, —N(CH₃)₂ or —N(C₂H₅)₂;
(f) R₂ is —COR₂ₐ and R₂ₐ is selected from ethyl, isopropyl, methoxy, ethoxy, propoxy, isopropoxy, isobutoxy, tert-butoxy, cyclopropyl, or —N(CH₃)₂;
(g) R₂ is —COR₂ₐ and the “C*” carbon center is in the S configuration; or R₂ is —SO₂R_{2b} and the “C*” carbon center is in the R configuration;
(h) R₃ is a heteroaryl group selected from the group consisting of pyridinyl, pyrimidinyl, pyrazinyl, pyridazinyl, thiazolyl, isothiazolyl, and thiadiazolyl, each optionally substituted with one or more substituents independently selected from the group consisting of methyl, bromo, chloro, fluoro, —CHF₂, hydroxyl, amino and —NHCH₂CH₂CO₂H;
(i) R₃ represents 6-fluoro-pyridin-2-yl, pyridine-2-yl, 3-hydroxy-pyridin-2-yl, 6-difluoromethyl-pyridin-2-yl, 2-amino-pyridin-3-yl, 2-carboxymethylamino-pyridin-3-yl, pyrimidin-4-yl, pyrimindin-2-yl, 2-chloro-pyrimidin-4-yl, thiazol-4-yl, 2-methyl-thiazol-4-yl, 2-chloro-thiazol-4-yl, thiazol-2-yl, thiazol-5-yl, thiazol-5-yl, 4-amino-thiazol-5-yl, pyrazine-2-yl, 5-methyl-pyrazin-2-yl, 3-chloro-pyrazin-2-yl, pyridazin-3-yl, 5-bromo-isothiazol-3-yl, isothiazol-3-yl, 4,5-dichloro-isothiazol-3-yl, or [1,2,5]thiadiazol-3-yl;
(j) R₃ is pyridine-2-yl, 2-amino-pyridin-3-yl, thiazol-5-yl, or 4-amino-thiazol-5-yl;
(k) R₃ is pyridine-2-yl, 2-amino-pyridin-3-yl, thiazol-5-yl, or 4-amino-thiazol-5-yl; or
(l) the compound is carbamic acid, N-[(2S)-7-cyano-1,2,3,4-tetrahydro-4-(2-pyridinylmethyl)cyclopent[b]indol-2-yl]-,1-methylethyl ester.

3-12. (canceled)

13. The method according to claim 1, further comprising administering at least one second active pharmaceutical ingredient, wherein:
(a) the second active pharmaceutical ingredient is a phosphodiesterase type-5 inhibitor;
(b) the second active pharmaceutical ingredient is a phosphodiesterase type-5 inhibitor, wherein the phosphodiesterase type-5 inhibitor is sildenafil, vardenafil, or tadalafil;
(c) the second active pharmaceutical ingredient is present in an amount of 5 mg, 15 mg, or 25 mg; or
(d) the first active ingredient and the second active ingredient are in a single dosage form.

14-16. (canceled)

17. The method according to claim 1, wherein:
(a) the compound of Formula I is present in an amount of about 0.5 mgs, to about 50 mgs;
(b) the compound of Formula I is administered once daily;
(c) the compound of Formula I is administered orally; or
(d) the compound of Formula I is administered in a gelatin capsule.

18-21. (canceled)

22. The method according to claim 1, wherein:
(a) the subject has BPH;
(b) the subject has moderate-to-severe lower urinary tract symptoms (LUTS) due to BPH;
(c) the subject has an enlarged prostate;
(d) the size or volume of the subject's prostate is reduced when compared to a baseline prior to the initiation of treatment;
(e) the size or volume of the subject's prostate is reduced by at least about 20 percent when compared to a baseline prior to the initiation of treatment;
(f) the level of prostate-specific antigen (PSA) in the subject is reduced when compared to a baseline prior to the initiation of treatment;
(g) the level of prostate-specific antigen (PSA) in the subject is reduced by 5, 10, 15, or 20 percent when compared to a baseline prior to the initiation of treatment;
(h) the lean body mass of the subject is increased when compared to a baseline prior to the initiation of treatment;
(i) the fat body mass of the subject is reduced when compared to a baseline prior to the initiation of treatment;
(j) the lower extremity muscle power of the subject is increased when compared to a baseline prior to the initiation of treatment;
(k) the fatigue experienced by the subject reduced when compared to a baseline prior to the initiation of treatment;
(l) the sexual dysfunction of the subject is reduced when compared to a baseline prior to the initiation of treatment; or
(m) the physical dysfunction of the subject is reduced when compared to a baseline prior to the initiation of treatment.

23-34. (canceled)

35. A method of treating the signs and symptoms of benign prostatic hyperplasia (BPH) in a subject comprising administering a therapeutically effect amount of the compound of Formula II or a pharmaceutically acceptable salt thereof to a subject in need thereof, wherein the compound has Formula II:

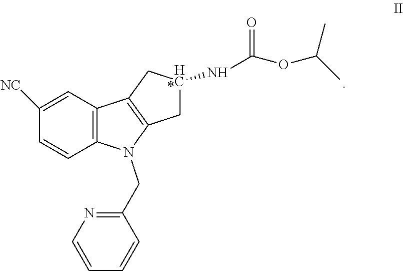

36. The method according to claim 35, further comprising administering at least one second active pharmaceutical ingredient, wherein:
(a) the second active pharmaceutical ingredient is a phosphodiesterase type-5 inhibitor;
(b) the second active pharmaceutical ingredient is a phosphodiesterase type-5 inhibitor, wherein the phosphodiesterase type-5 inhibitor is sildenafil, vardenafil, or tadalafil; or
(c) the second active pharmaceutical ingredient is present in an amount of about 5 mg, 15 mg, or 25 mg;

37-39. (canceled)

40. The method according to claim 35, wherein:
(a) the compound of Formula II is present in an amount of about 5 mg, 15 mg, or 25 mg;
(b) the compound of Formula II is administered once daily;
(c) the compound of Formula II is administered orally;
(d) the compound of Formula II is administered in a gelatin capsule; or
(e) the compound of formula II is in the (2S) configuration.

41-43. (canceled)

44. The method according to any one of claims 35-43, wherein:
(a) the subject has BPH;
(b) the subject has moderate-to-severe lower urinary tract symptoms (LUTS) due to BPH;
(c) the subject has an enlarged prostate;
(d) the size or volume of the subject's prostate is reduced when compared to a baseline prior to the initiation of treatment;
(e) the size or volume of the subject's prostate is reduced by least about 20 percent when compared to a baseline prior to the initiation of treatment;
(f) the level of prostate-specific antigen (PSA) in the subject is reduced when compared to a baseline prior to the initiation of treatment;
(g) the level of prostate-specific antigen (PSA) in the subject is reduced by 5, 10, 15, or 20 percent when compared to a baseline prior to the initiation of treatment
(h) the lean body mass of the subject is increased when compared to a baseline prior to the initiation of treatment;
(i) the fat body mass of the subject is reduced when compared to a baseline prior to the initiation of treatment;
(j) the lower extremity muscle power of the subject is increased when compared to a baseline prior to the initiation of treatment;
(k) the fatigue experienced by the subject reduced when compared to a baseline prior to the initiation of treatment;
(l) the sexual dysfunction of the subject is reduced when compared to a baseline prior to the initiation of treatment;
(m) the physical dysfunction of the subject is reduced when compared to a baseline prior to the initiation of treatment.

45-57. (canceled)

58. A pharmaceutical composition comprising a compound of formula I or II and a PDE5 inhibitor and pharmaceutically acceptable excipients in a fixed unit combination oral dosage from.

59. The pharmaceutical composition according to claim 58, comprising a compound of formula II and tadalafil wherein the fixed unit combination dosage form is a capsule or a tablet.

60. The pharmaceutical composition according to claim 59 wherein the compound of formula II is carbamic acid, N-[(2S)-7-cyano-1,2,3,4-tetrahydro-4-(2-pyridinylmethyl)cyclopent[b]indol-2-yl]-, 1-methylethyl ester.

61. A method of treating a patient having signs or symptoms of BPH comprising administering a pharmaceutically effective amount of a first compound selected from carbamic acid, N-[(2S)-7-cyano-1,2,3,4-tetrahydro-4-(2-pyridinylmethyl)cyclopent[b]indol-2-yl]-, 1-methylethyl ester and, optionally, a second compound selected from tadalafil and having the first compound at a dosage strength of 1 to 25 mgs and having the optional second compound at a dosage strength of 1 to 10 mgs.

62. The method according to claim 61 wherein the first compound, in a single oral dosing, has at least one of a Cₘₐₓ(ng/mL) of between 25 to 123; a tₘₐₓ(h) of about 4-5 hr; a t_{1/2} (h) of about 25-28 h and an AUC₍₀₋₎(ng h/mL) of about 400 to 2200.

63. The method according to claim 61, wherein the first compound and the second compound are in a fixed-dose combination dosage form.
